(19) United States
(12) Patent Application Publication
(10) Pub. No.: US 2025/0092052 A1
(43) Pub. Date: Mar. 20, 2025

(54) HETEROARYL DERIVATIVE COMPOUNDS, AND USES THEREOF

(71) Applicant: VORONOI INC., Incheon (KR)

(72) Inventors: Seohyun JO, Incheon (KR); Daekwon KIM, Incheon (KR)

(73) Assignee: VORONOI INC., Incheon (KR)

(21) Appl. No.: 18/887,279

(22) Filed: Sep. 17, 2024

(30) Foreign Application Priority Data
Sep. 18, 2023 (KR) 10-2023-0124264

(51) Int. Cl. C07D 487/04; A61K 31/438; A61K 31/5025; A61K 31/5377; C07D 519/00

(52) U.S. Cl. CPC C07D 487/04; A61K 31/438; A61K 31/5025; A61K 31/5377; C07D 519/00

(57) ABSTRACT

The present disclosure relates to heteroaryl derivatives and their uses thereof. The heteroaryl derivatives of the present disclosure exhibit excellent inhibitory activity against PKMYT1 and/or CCNE1, can be useful as therapeutic agents for diseases related to the overactivation, amplification, or overexpression of PKMYT1 and/or CCNE1.

### CROSS-REFERENCE TO RELATED APPLICATION

This application claims priority to Korean Patent Application No. 10-2023-0124264 filed Sep. 18, 2023, the entire disclosure of which is incorporated herein by reference.

### TECHNICAL FIELD

The present disclosure relates to heteroaryl derivative compounds and their medicinal uses. Specifically, the present disclosure relates to heteroaryl derivative compounds have inhibitory activity against PKMYT1 and/or CCNE1.

### BACKGROUND ART

Protein kinases function as molecular switches involved in signal transduction pathways, where the transition between active and inactive states of target proteins by kinases must be smoothly regulated within the cell. If this transition between active and inactive states is abnormally regulated, it can lead to either excessive activation or inactivation of cellular signaling, ultimately causing uncontrolled cell division and proliferation. Especially, abnormal activation due to mutations, amplification, and/or overexpression of protein kinase genes can lead to the occurrence and progression of various tumors, as well as play a critical role in the pathogenesis of various diseases, including inflammatory diseases, neurodegenerative disorders, and autoimmune disease.

In particular, PKMYT1 (MYT1; membrane-associated tyrosine and threonine specific cdc2 inhibitory kinase) is a member of the Wee1 kinase family and acts as a key regulator of the G2 checkpoint in the cell cycle, along with Wee1, controlling the entry into mitosis. When DNA damage occurs in cancer cells, PKMYT1 repairs (DDR; DNA Damage Repair) damaged DNA through the G2 checkpoint (Molecules, 2017, 22(12), 2045). Therefore, by inhibiting PKMYT1, it is possible to suppress the function of the G2/M checkpoint in cancer cells, induce mitotic catastrophe, and thereby inhibit the activity of cancer cells (Journal of Hematology & Oncology, 2020, 13, 126).

In many patients with solid tumors, chemotherapy is not responsive, which is due to cell cycle regulation and DDR of cancer cells. Additionally, PKMYT1 is overexpressed in solid tumors, including non-small cell lung cancer (NSCLC), colorectal cancer (CRC), gastric cancer, hepatocellular carcinoma, glioblastoma, and breast cancer (Cell Proliferation, 2020, 53, e12741). In NSCLC patients, overexpression of PKMYT1 has been shown to promote cancer progression and metastasis, leading to a poor prognosis (Eur Rev Med Pharmacol Sci., 2019, 23(10), 4210-4219).

Synthetic lethality is a phenomenon in which cell death is induced as a complex resulting of mutation, suppression, or overexpression of two or more genes, whereas a defect in only one gene does not cause cell death. Synthetic lethality is gaining attention as a promising strategy for developing novel anticancer drugs, as it can specifically target various cellular defects, including DNA repair, cell cycle regulation, and metabolic processes, to induce selective cancer cell death.

Overexpression of CCNE1, a cell cycle regulator, induces DNA damage during the DNA replication process. At this point, cells are arrested in the G2 phase and undergo a DNA repair process to correct damaged DNA. PKMYT1, a key regulator of the G2 checkpoint, has increased expression and activity by CCNE1 overactivation, amplification, or overexpression. While inhibition of PKMYT1 alone does not induce cell death, inhibiting PKMYT1 in cancer cells with increased DNA damage due to CCNE1 overactivation, amplification, or overexpression can induce synthetic lethality through cell death.

As described above, PKMYT1 exhibits CCNE1 dependent activity, which can be categorized into intrinsic dependent activity, which arises from the overactivation, amplification, or overexpression of the CCNE1 gene; and induced dependent activity, which occurs due to external factors such as chemotherapy that led to overactivation, amplification, or overexpression of CCNE1.

Accordingly, there is an increasing demand for novel compounds that can be useful utilized in the treatment of PKMYT1-related diseases and disorders caused by the overactivation, amplification, or overexpression of CCNE1.

### PRIOR ART DOCUMENTS

### Non-Patent Document

Molecules, 2017, 22(12), 2045
Journal of Hematology & Oncology, 2020, 13, 126
Cell Proliferation, 2020, 53, e12741
Eur Rev Med Pharmacol Sci., 2019, 23(10), 4210-4219

### DISCLOSURE

### Technical Problem

It is object of the present disclosure is to provide heteroaryl derivatives of novel structure, a stereoisomer thereof, or a pharmaceutically acceptable salt thereof.

It is another object of the present disclosure is to provide a method for preparing the above heteroaryl derivative compounds.

It is another object of the present disclosure to provide the medicinal use of the above heteroaryl derivative compounds, specifically to provide a pharmaceutical composition for the treatment or prevention of diseases associated with the overactivation, amplification, or overexpression of PKMYT1 and/or CCNE1, comprising the heteroaryl derivative compounds as an active ingredient.

It is another object of the present disclosure is to provide a use for treating or preventing a disease associated with PKMYT1 and/or CCNE1 overactivation, amplification, or overexpression using the compound, or a method for treating or preventing a disease associated with PKMYT1 and/or CCNE1 overactivation, amplification, or overexpression comprising a step of administering the compound.

### Technical Solution

In order to achieve the above object, as a result of research efforts of the present inventors have completed the invention by confirming that the heteroaryl derivative compounds represented by Chemical Formula 1, 2, 3, or 4, as described below, inhibit the proliferation of cells in which PKMYT1 and/or CCNE1 are overactivated, amplified, or overexpressed.

### Heteroaryl Derivative Compounds

The present disclosure provides a compound represented by the following Chemical Formula 1, a stereoisomer thereof, or a pharmaceutically acceptable salt thereof:

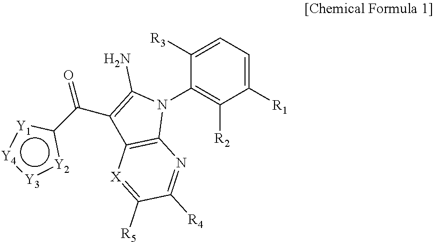

In Chemical Formula 1 above,
R₁ to R₃ are each independently —C₁₋₆ alkyl or —OH;
R₄ and R₅ are each independently —H, —C₁₋₆ alkyl, —C₂₋₆ alkenyl, —C₂₋₆ alkynyl, —C₁₋₆ aminoalkyl, —C₁₋₆ hydroxyalkyl, —C₁₋₆ haloalkyl, —CN, —NH(C₁₋₆ alkyl), —N(C₁₋₆ alkyl)(C₁₋₆ alkyl), —O—(C₁₋₆ alkyl), 3-6 membered cycloalkyl, 3-12 membered heterocycloalkyl, phenyl, or 5-6 membered heteroaryl {wherein one or more H atoms in the ring of the 3-6 membered cycloalkyl, 3-12 membered heterocycloalkyl, phenyl, or 5-6 membered heteroaryl may be substituted with —C₁₋₆ alkyl}.
X is CH or N;
Y₁ and Y₂ are each independently NH, N, or —CR_{Y1};
Y₃ and Y₄ are each independently N or CR_{Y2}, or Y₃ and Y₄ are bonded together to form an 8-9 membered aromatic fused ring {wherein the 8-9 membered aromatic fused ring may include one or more N, O, or S atoms within the ring; and one or more H atoms in the 8-9 membered aromatic fused ring may be substituted with —C₁₋₆ alkyl, —C₁₋₆ aminoalkyl, —C₁₋₆ hydroxyalkyl, —C₁₋₆ haloalkyl, —(CH₂)m-Rₐ, —NH—R_{b}, —O—R_{c}, or -halo};
R_{Y1} and R_{Y2} are each independently —H, —C₁₋₆ alkyl, or —C₁₋₆ alkenyl;
m is 0, 1, 2, 3, or 4;
Rₐ is —S(═O)₂—C₁₋₆ alkyl, —P(═O)—(C₁₋₆ alkyl)(C₁₋₆ alkyl), 3-12 membered heterocycloalkyl, or —C(═O)-(3-6 membered cycloalkyl) {wherein one or more H atoms in the 3-12 membered heterocycloalkyl or —C(═O)-(3-6 membered cycloalkyl) ring may be substituted with —C₁₋₆ alkyl, —O—C₁₋₆ alkyl, —C₁₋₆ haloalkyl, or -halo}.
R_{b} and R_{c} are each independently —C₁₋₆ alkyl, —C₁₋₆ haloalkyl, —S(═O)₂—C₁₋₆ alkyl, 3-6 membered heterocycloalkyl, or 5-6 membered heteroaryl {wherein one or more H atoms in the 3-6 membered heterocycloalkyl or 5-6 membered heteroaryl ring may be substituted with —C₁₋₆ alkyl, —O—C₁₋₆ alkyl, —C₁₋₆ haloalkyl, or -halo}.

Also, the present disclosure provides compounds represented by Chemical Formula 2, a stereoisomer thereof, or a pharmaceutically acceptable salt thereof.

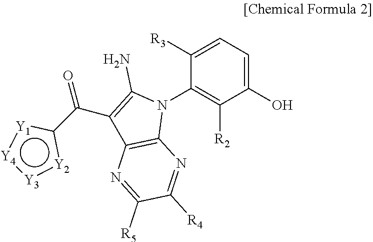

In Chemical Formula 2 above,
R₂ and R₃ are each independently —C₁₋₆ alkyl;
R₄ and R₅ are each independently —H, —C₁₋₆ alkyl, —C₂₋₆ alkenyl, —C₂₋₆ alkynyl, —C₁₋₆ aminoalkyl, —C₁₋₆ hydroxyalkyl, —C₁₋₆ haloalkyl, —CN, —NH(C₁₋₆ alkyl), —N(C₁₋₆ alkyl)(C₁₋₆ alkyl), —O—(C₁₋₆ alkyl), 3-6 membered cycloalkyl, 3-12 membered heterocycloalkyl, phenyl, or 5-6 membered heteroaryl {wherein one or more H atoms in the ring of the 3-6 membered cycloalkyl, 3-12 membered heterocycloalkyl, phenyl, or 5-6 membered heteroaryl may be substituted with —C₁₋₆ alkyl}.
Y₁ is NH or —CR_{Y1};
Y₂ is N or —CR_{Y1};
Y₃ and Y₄ are each independently N or CR_{Y2}, or Y₃ and Y₄ are bonded together to form an 8-9 membered aromatic fused ring {wherein the 8-9 membered aromatic fused ring may include one or more N, O, or S atoms within the ring; and one or more H atoms in the 8-9 membered aromatic fused ring may be substituted with —C₁₋₆ alkyl, —C₁₋₆ aminoalkyl, —C₁₋₆ hydroxyalkyl, —C₁₋₆ haloalkyl, —(CH₂)m-Rₐ, —NH—R_{b}, —O—R_{c}, or -halo};
R_{Y1} and R_{Y2} are each independently —H, —C₁₋₆ alkyl, or —C₁₋₆ alkenyl;
m is 0, 1, 2, 3, or 4;
Rₐ is —S(═O)₂—C₁₋₆ alkyl, —P(═O)—(C₁₋₆ alkyl)(C₁₋₆ alkyl), 3-12 membered heterocycloalkyl, or —C(═O)-(3-6 membered cycloalkyl) {wherein one or more H atoms in the 3-12 membered heterocycloalkyl or —C(═O)-(3-6 membered cycloalkyl) ring may be substituted with —C₁₋₆ alkyl, —O—C₁₋₆ alkyl, —C₁₋₆ haloalkyl, or -halo}.
R_{b} and R_{c} are each independently —C₁₋₆ alkyl, —C₁₋₆ haloalkyl, —S(═O)₂—C₁₋₆ alkyl, 3-6 membered heterocycloalkyl, or 5-6 membered heteroaryl {wherein one or more H atoms in the 3-6 membered heterocycloalkyl or 5-6 membered heteroaryl ring may be substituted with —C₁₋₆ alkyl, —O—C₁₋₆ alkyl, —C₁₋₆ haloalkyl, or -halo}.

Also, the present disclosure provides compounds represented by Chemical Formula 3, a stereoisomer thereof, or a pharmaceutically acceptable salt thereof.

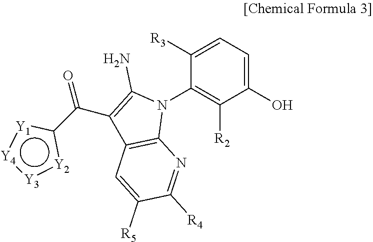

In Chemical Formula 3 above,
R₂ and R₃ are each independently —C₁₋₆ alkyl;
R₄ and R₅ are each independently —H, —C₁₋₆ alkyl, —C₂₋₆ alkenyl, —C₂₋₆ alkynyl, —C₁₋₆ aminoalkyl, —C₁₋₆ hydroxyalkyl, —C₁₋₆ haloalkyl, —CN, —NH(C₁₋₆ alkyl), —N(C₁₋₆ alkyl)(C₁₋₆ alkyl), —O—(C₁₋₆ alkyl), 3-6 membered cycloalkyl, 3-12 membered heterocycloalkyl, phenyl, or 5-6 membered heteroaryl {wherein one or more H atoms in the ring of the 3-6 membered cycloalkyl, 3-12 membered heterocycloalkyl, phenyl, or 5-6 membered heteroaryl may be substituted with —C₁₋₆ alkyl}.

Y₁ is NH or CR_{Y1};
Y₂ is N or CR_{Y1};
Y₃ and Y₄ are each independently N or CR_{Y2}, or Y₃ and Y₄ are bonded together to form an 8-9 membered aromatic fused ring {wherein the 8-9 membered aromatic fused ring may include one or more N, O, or S atoms within the ring; and one or more H atoms in the 8-9 membered aromatic fused ring may be substituted with —C₁₋₆ alkyl, —C₁₋₆ aminoalkyl, —C₁₋₆ hydroxyalkyl, —C₁₋₆ haloalkyl, —(CH₂)m-Rₐ, —NH—R_{b}, —O—R_{c}, or -halo};
R_{Y1} and R_{Y2} are each independently —H, —C₁₋₆ alkyl, or —C₁₋₆ alkenyl;
m is 0, 1, 2, 3, or 4;
Rₐ is —S(═O)₂—C₁₋₆ alkyl, —P(═O)—(C₁₋₆ alkyl)(C₁₋₆ alkyl), 3-12 membered heterocycloalkyl, or —C(═O)-(3-6 membered cycloalkyl) {wherein one or more H atoms in the 3-12 membered heterocycloalkyl or —C(═O)-(3-6 membered cycloalkyl) ring may be substituted with —C₁₋₆ alkyl, —O—C₁₋₆ alkyl, —C₁₋₆ haloalkyl, or -halo}; and
R_{b} and R_{c} are each independently —C₁₋₆ alkyl, —C₁₋₆ haloalkyl, —S(═O)₂—C₁₋₆ alkyl, 3-6 membered heterocycloalkyl, or 5-6 membered heteroaryl {wherein one or more H atoms in the 3-6 membered heterocycloalkyl or 5-6 membered heteroaryl ring may be substituted with —C₁₋₆ alkyl, —O—C₁₋₆ alkyl, —C₁₋₆ haloalkyl, or -halo}.

Also, the present disclosure provides compounds represented by Chemical Formula 4, a stereoisomer thereof, or a pharmaceutically acceptable salt thereof.

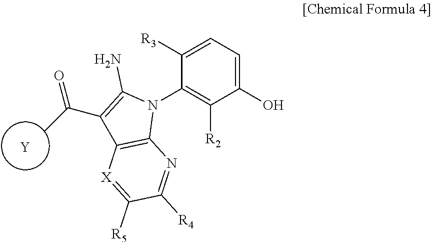

R₂ and R₃ are each independently —C₁₋₆ alkyl;
R₄ and R₅ are each independently —H, —C₁₋₆ alkyl, —C₂₋₆ alkenyl, —C₂₋₆ alkynyl, —C₁₋₆ aminoalkyl, —C₁₋₆ hydroxyalkyl, —C₁₋₆ haloalkyl, —CN, —NH(C₁₋₆ alkyl), —N(C₁₋₆ alkyl)(C₁₋₆ alkyl), —O—(C₁₋₆ alkyl), 3-6 membered cycloalkyl, 3-12 membered heterocycloalkyl, phenyl, or 5-6 membered heteroaryl {wherein one or more H atoms in the ring of the 3-6 membered cycloalkyl, 3-12 membered heterocycloalkyl, phenyl, or 5-6 membered heteroaryl may be substituted with —C₁₋₆ alkyl}.
X is CH or N;

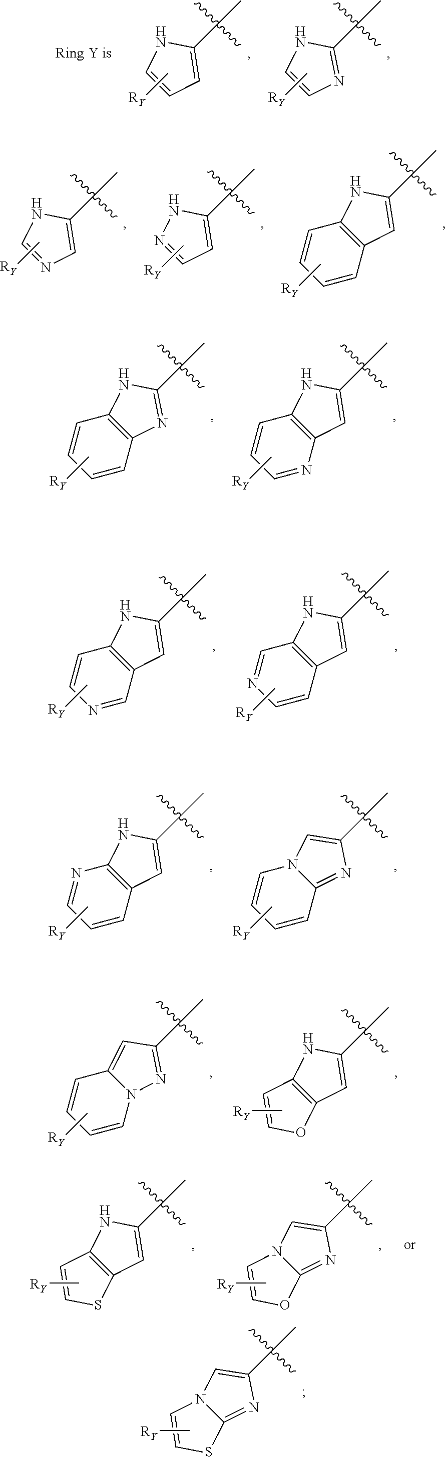

R_{Y} is —H, —C₁₋₆ alkyl, —C₁₋₆ aminoalkyl, —C₁₋₆ hydroxyalkyl, —C₁₋₆ haloalkyl, —(CH₂)m-Rₐ, —NH—Rb, —O—R_{c}, or -halo;
m is 0, 1, 2, 3, or 4;
Rₐ is —S(═O)₂—C₁₋₆ alkyl, —P(═O)—(C₁₋₆ alkyl)(C₁₋₆ alkyl), 3-12 membered heterocycloalkyl, or —C(═O)-(3-6 membered cycloalkyl) {wherein one or more H atoms in the 3-12 membered heterocycloalkyl or —C(═O)-(3-6 membered cycloalkyl) ring may be substituted with —C₁₋₆ alkyl, —O—C₁₋₆ alkyl, —C₁₋₆ haloalkyl, or -halo}.
R_{b} and R_{c} are each independently —C₁₋₆ alkyl, —C₁₋₆ haloalkyl, —S(═O)₂—C₁₋₆ alkyl, 3-6 membered heterocycloalkyl, or 5-6 membered heteroaryl {wherein one or more H atoms in the 3-6 membered heterocycloalkyl or 5-6 membered heteroaryl ring may be substituted with —C₁₋₆ alkyl, —O—C₁₋₆ alkyl, —C₁₋₆ haloalkyl, or -halo}.

According to an embodiment of the present disclosure, the compounds represented by above Chemical Formula 1, 2, 3, or 4 may be selected from the group consisting of the compounds listed in Table 1 below.

In the present disclosure, unless otherwise specified, “alkyl” may mean a straight or branched chain, acyclic, cyclic, or a saturated hydrocarbon of which they are bonded. For example, “C₁₋₆alkyl” may mean an alkyl containing 1 to 6 carbon atoms. The acyclic alkyl may include, for example, methyl, ethyl, n-propyl, n-butyl, isopropyl, sec-butyl, isobutyl, tert-butyl, and the like, but is not limited to. The cyclic alkyl may be used interchangeably with “cycloalkyl” herein, and as an example, may include cyclopropyl, cyclobutyl, cyclopentyl, cyclohexyl, cycloheptyl, cyclooctyl, and the like, but is not limited to.

In the present disclosure, “alkoxy” may mean —(O-alkyl) as an alkyl ether group, wherein alkyl is same as defined above. For example, “C₁₋₆alkoxy” may mean alkoxy containing C₁₋₆alkyl, that is, —(O—C₁₋₆alkyl), and as an example, alkoxy may include methoxy, ethoxy, n-propoxy, isopropoxy, n-butoxy, isobutoxy, sec-butoxy, tert-butoxy, and the like, but is not limited thereto.

In the present disclosure, “halo” may be F, Cl, Br, or I.

In the present disclosure, “haloalkyl” may mean a straight-chain or branched-chain alkyl(hydrocarbon) having one or more halo-substituted carbon atoms as defined herein. Examples of the haloalkyl may include methyl, ethyl, propyl, isopropyl, isobutyl, or n-butyl independently substituted with one or more halogens, such as F, Cl, Br, or I, but are not limited thereto.

In the present disclosure, “hydroxyalkyl” may mean straight-chain or branched-chain alkyl(hydrocarbon) having a carbon atom substituted with hydroxy (OH). Examples of the hydroxyalkyl may include each methyl independently substituted with —OH, methyl, ethyl, propyl, isopropyl, isobutyl, or n-butyl, but are not limited thereto.

In the present disclosure, “aminoalkyl” may mean straight-chain or branched-chain alkyl(hydrocarbon) having a carbon atom substituted with amino (NR′R″). Here, R′ and R″ may each independently be selected from the group consisting of hydrogen and C₁₋₆alkyl, and the selected R′ and R″ may each independently be substituted or unsubstituted.

In the present disclosure, “cyanoalkyl” may mean straight-chain or branched-chain alkyl(hydrocarbon) having a carbon atom substituted with cyano (CN).

In the present disclosure, “cycloalkyl” may mean a hydrocarbon ring that does not contain heteroatoms (such as N, O, P, P(═O), or S) within the ring and may be saturated or partially unsaturated. In cases where it is unsaturated, it may be referred to as cycloalkenyl. Unless otherwise stated, cycloalkyl can be a single ring or a polycyclic structure such as a spiro ring, bridged ring, or fused ring.

In the present disclosure, “heterocycloalkyl” may mean a ring containing at least one selected from N, O, P, P(═O), and S in the ring, and may be saturated or partially unsaturated. Here, when unsaturated, it may be referred to as a heterocycloalkene. Unless otherwise stated, a heterocycloalkyl can be a monocyclic ring, or a polycyclic ring, such as a spiro ring, a bridged ring, or a fused ring. In addition, “3- to 12-membered heterocycloalkyl” may mean a heterocycloalkyl containing 3 to 12 atoms forming a ring, and as an example, heterocycloalkyl may include pyrrolidine, piperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyron, tetrahydrofuran, tetrahydrothiophene, quinuclidine, tropane, 2-azaspiro[3.3]heptane, (1r,5s)-3-azabicyclo[3.2.1]octane, (1s,4s)-2-azabicyclo[2.2.2]octane, or (1r,4r)-2-oxa-5-azabicyclo[2.2.2]octane, and the like, but is not limited thereto.

In the present disclosure, “arene” may mean an aromatic hydrocarbon ring. The arene may be a monocyclic arene or a polycyclic arene. The number of ring-forming carbon atoms of the arene may be 5 or more and 30 or less, 5 or more and 20 or less, or 5 or more and 15 or less. Examples of the arene may include benzene, naphthalene, fluorene, anthracene, phenanthrene, bibenzene, terbenzene, quarterbenzene, quinquebenzene, sexibenzene, triphenylene, pyrene, benzofluoranthene, chrysene, and the like, but are not limited thereto. In the present specification, a residue obtained by removing one hydrogen atom from the above “arene” is referred to as “aryl”.

In the present disclosure, the “heteroarene” may be a ring including one or more of O, N, P, Si, and S as a heterogeneous element. The number of ring-forming carbon atoms of heteroarene may be 2 or more and 30 or less, or 2 or more and 20 or less. The heteroarene may be a monocyclic heteroarene or a polycyclic heteroarene. The polycyclic heteroarenes may have, for example, a bicyclic or tricyclic structure. Examples of the heteroarene may include thiophene, purine, pyrrole, pyrazole, imidazole, thiazole, oxazole, isothiazole, oxadiazole, triazole, pyridine, pyridin-2-one, pyridin-3-one, pyridin-4-one, bipyridyl, triazine, acridyl, pyridazine, pyrazine, quinoline, quinazoline, quinoxaline, phenoxazine, phthalazine, pyrimidine, pyrido pyrimidine, pyrido pyrazine, pyrazino pyrazine, isoquinoline, indole, carbazole, imidazopyridazine, imidazopyridine, imidazopyrimidine, pyrazolopyrimidine, imidazopyrazine, pyrazolopyridine, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, isoxazole, oxadiazole, thiadiazole, benzothiazole, tetrazole, phenothiazine, dibenzosilol, dibenzofuran, and the like, but are not limited thereto. In one embodiment of the present disclosure, the heteroarene may also include a bicyclic heterocyclo-arene containing an arene ring fused to a heterocycloalkyl ring or a heteroarene fused to a cycloalkyl ring. In the present specification, a residue obtained by removing one hydrogen atom from the “heteroarene” is referred to as “heteroaryl”.

In the present disclosure, “stereoisomer” means a compound of the present disclosure having the same chemical formula or molecular formula but sterically different. Stereoisomers in this specification include optical isomers, enantiomers, diastereomers, cis/trans isomers, rotamers, and atropisomers, and each of these isomers, racemates, and their mixtures are also within the scope of the present disclosure. For example, the compounds represented by Chemical Formula 1, 2, 3, or 4 of the present disclosure may include these stereoisomers, as the stereochemical structure is not specified. Unless otherwise stated, a solid-line bond ( [symbol] ) connected to an asymmetric carbon atom may include a wedge-shaped solid-line bond ( [symbol] ) or a wedge-shaped dotted-line bond ( [symbol] ) representing an absolute arrangement of the stereocenter.

The compound of Chemical Formula 1, 2, 3, or 4 of the present disclosure may exist in the form of a “pharmaceutically acceptable salt”. Accordingly, the scope of the compounds of the present disclosure includes pharmaceutically acceptable salts of the compounds represented by Chemical Formula 1, 2, 3, or 4. The term “pharmaceutically acceptable salt” refers to any organic acid or inorganic acid addition salt of the compound whose side effects do not reduce the beneficial efficacy of the compound represented by Chemical Formula 1, 2, 3, or 4 at concentrations having an effective action that is relatively non-toxic and harmless to a patient.

In particular, the pharmaceutically acceptable salts may be acid addition salts formed by free acids. Wherein, acid addition salts can be obtained from inorganic acids such as hydrochloric acid, nitric acid, phosphoric acid, sulfuric acid, hydrobromic acid, hydroiodic acid, nitrous acid, and phosphorous acid; non-toxic organic acids such as aliphatic mono- and dicarboxylates, phenyl-substituted alkanoates, hydroxy alkanoates and alkanedioates, aromatic acids, aliphatic and aromatic sulfonic acids; organic acids such as trifluoroacetic acid, acetate, benzoic acid, citric acid, lactic acid, maleic acid, gluconic acid, methanesulfonic acid, p-toluenesulfonic acid, tartaric acid, and fumaric acid.

The types of pharmaceutically acceptable salts may include sulfates, sulfites, nitrates, phosphates, pyrophosphates, chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, benzoates, phthalates, benzenesulfonates, toluenesulfonates, phenylacetates, phenylpropionates, phenylbutyrates, citrates, lactates, glycolates, malates, tartrates, and mandelates, and the like.

The above acid addition salts can be prepared by conventional methods, for example, a derivative of the compounds of Chemical Formula 1, 2, 3, or 4 can be dissolved in an organic solvent such as methanol, ethanol, acetone, methylene chloride, or acetonitrile, followed by the addition of an organic or inorganic acid, with the resulting precipitate can then be filtered and dried, or alternatively, can be prepared by crystallization under organic solvents after removing the solvent and excess acid by vacuum distillation, followed by drying.

Additionally, the pharmaceutically acceptable salts may include salts obtained using a base or metal salts. Example of a metal salt, such as an alkali metal or alkaline earth metal salt, can be obtained by dissolving the compound in an excess of an alkali metal hydroxide or alkaline earth metal hydroxide solution, filtering the undissolved compound salt, and then evaporating and drying the filtrate. Alkali metal salts such as sodium, potassium, or calcium salts may be pharmaceutically suitable. Moreover, the corresponding salts can be obtained by reacting the alkali metal or alkaline earth metal salts with an appropriate silver salt (e.g., silver nitrate) and may be prepared through a method for preparing a salt known to the art.

### Use of Heteroaryl Derivative Compounds

The present disclosure provides the use of a compound represented by the following Chemical Formula 1, 2, 3, or 4, a stereoisomer thereof, or a pharmaceutically acceptable salt thereof.

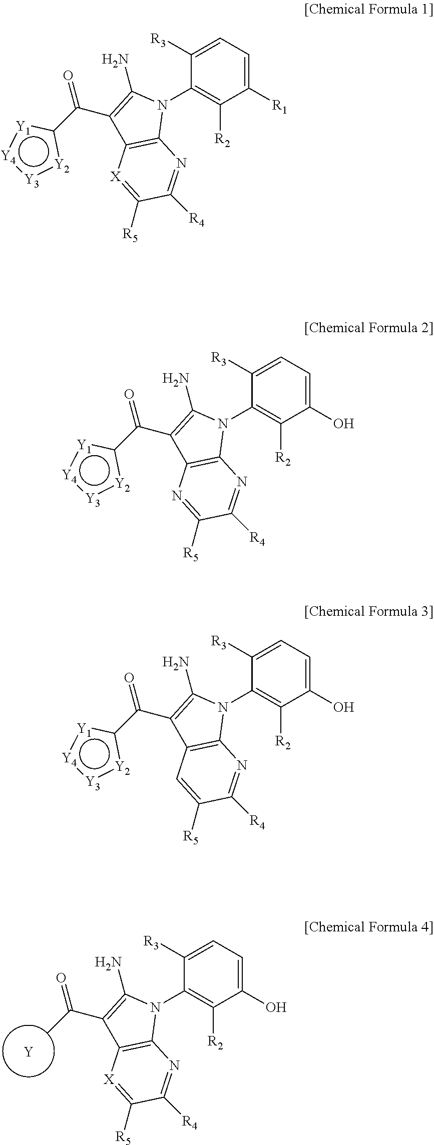

The Chemical Formula 1, 2, 3, or 4 are as defined above.

The compounds represented by Chemical Formula 1, 2, 3, or 4 of the present disclosure, a stereoisomer thereof, or a pharmaceutically acceptable salt thereof exhibit inhibitory activity against various kinases.

According to one embodiment of the present disclosure, the heteroaryl derivatives represented by Chemical Formula 1, 2, 3, or 4 exhibit high inhibitory activity against PKMYT1 and have been found to suppress the proliferation of cells in which PKMYT1 is activated. Additionally, the heteroaryl derivatives represented by Chemical Formula 1, 2, 3, or 4 of the present disclosure also demonstrate significant inhibitory activity against cells with CCNE1 overactivation, amplification, or overexpression. Therefore, these compounds can be effectively used for the treatment or prevention of diseases associated with the overactivation, amplification, or overexpression of PKMYT1 and/or CCNE1.

The diseases associated with the overactivation, amplification, or overexpression of PKMYT1 and/or CCNE1 may include, for example, cancer. The cancer may be a malignant tumor associated with diseases or disorders related to PKMYT1, including abnormalities that activate PKMYT1, or it may be a malignant tumor characterized by the overactivation, amplification, or overexpression of CCNE1. The above cancer may be either a solid tumor or a hematologic malignancy, and it includes both primary and metastatic cancers.

As a specific example, the diseases associated with the overactivation, amplification, or overexpression of PKMYT1 and/or CCNE1 may include uterine cancer, endometrial cancer, uterine papillary serous carcinoma (UPSC), uterine carcinosarcoma, cervical cancer, uterine corpus cancer, uterine corpus endometrial carcinoma, ovarian cancer, serous ovarian cancer, high grade serous ovarian cancer (HGSOC), breast cancer, triple-negative breast cancer (TNBC), pancreatic cancer, pancreatic ductal adenocarcinoma (PDAC), lung cancer, non-small cell lung cancer (NSCLC), lung squamous cell carcinoma, colorectal cancer (CRC), gastric cancer, esophageal cancer, rectal cancer, leukemia, chronic myeloid leukemia, soft tissue sarcoma, brain cancer, liver cancer, liver hepatocellular carcinoma (LIHC), bladder cancer, bone cancer, osteosarcoma, Ewing sarcoma, chondrosarcoma, prostate cancer, testicular cancer, kidney cancer, kidney renal papillary cell carcinoma (KIRP), kidney renal clear cell carcinoma (KIRC), head and neck cancer, lymphoma, adenoid cystic carcinoma (ACC), skin cancer, melanoma, thymoma, glioblastoma, glioma, and brain lower-grade glioma (LGG), and may be selected from one or more of the group consisting of the above diseases, but are not limited thereto.

According to one embodiment of the present disclosure, the present disclosure provides a pharmaceutical composition for the treatment or prevention of diseases associated with the overactivation, amplification, or overexpression of PKMYT1 and/or CCNE1, comprising as an active ingredient a compound represented by Chemical Formula 1, 2, 3, or 4, its stereoisomer, or a pharmaceutically acceptable salt thereof. Specifically, the diseases associated with the overactivation, amplification, or overexpression of PKMYT1 and/or CCNE1 may be cancer. The types of cancer are as described above.

The pharmaceutical composition of the present disclosure may further include one or more active ingredients exhibiting the same or similar efficacy in addition to the compound represented by Chemical Formula 1, 2, 3, or 4, the stereoisomer thereof, or the pharmaceutically acceptable salt thereof.

The pharmaceutical composition of the present disclosure can be used for clinical administration and can be prepared to be administered in various oral and parenteral dosage forms.

Furthermore, in one embodiment of the present disclosure, the invention provides the use of the compound represented by Chemical Formula 1, 2, 3, or 4, the stereoisomer thereof, or the pharmaceutically acceptable salt thereof, for the manufacture of a medicament for the treatment or prevention of diseases associated with PKMYT1 and/or CCNE1 overactivation, amplification, or overexpression. Specifically, the diseases associated with PKMYT1 and/or CCNE1 overactivation, amplification, or overexpression may be cancer. The types of cancer are as mentioned above.

In addition, in one embodiment of the present disclosure, the invention provides the use of the compound represented by Chemical Formula 1, 2, 3, or 4, the stereoisomer thereof, or the pharmaceutically acceptable salt thereof, for the manufacture of a medicament for the treatment or prevention of cancer. The types of cancer are as mentioned above.

Furthermore, in one embodiment of the present disclosure, a method for treating or preventing a disease related to PKMYT1 and/or CCNE1 overactivation, amplification, or overexpression is provided, comprising administering a therapeutically effective amount of a compound represented by Chemical Formula 1, 2, 3, or 4, the stereoisomer thereof, or the pharmaceutically acceptable salt thereof, to a subject in need thereof. The subject may be a mammal including a human. Specifically, the disease related to PKMYT1 and/or CCNE1 overactivation, amplification, or overexpression may be cancer. The types of cancer are as mentioned above.

In addition, in one embodiment of the present disclosure, a method for treating or preventing cancer is provided, comprising administering a therapeutically effective amount of a compound represented by Chemical Formula 1, 2, 3, or 4, the stereoisomer thereof, or the pharmaceutically acceptable salt thereof, to a subject in need thereof. The types of cancer are as mentioned above.

Furthermore, in one embodiment of the present disclosure, a method for inhibiting PKMYT1 and/or CCNE1 is provided, comprising administering a therapeutically effective amount of a compound represented by Chemical Formula 1, 2, 3, or 4, the stereoisomer thereof, or the pharmaceutically acceptable salt thereof, to a subject in need thereof.

The term “therapeutically effective amount” used in the present disclosure refers to the amount of a compound represented by Chemical Formula 1, 2, 3, or 4 that is effective for the treatment or prevention of a disease related to the overactivation, amplification, or overexpression of PKMYT1 and/or CCNE1. Specifically, “therapeutically effective amount” means an amount sufficient to treat a disease with a reasonable benefit/risk ratio applicable to medical treatment, and the effective dose level may be determined by factors including individual type and severity, age, sex, disease type, drug activity, drug sensitivity, administration time, administration route and discharge rate, treatment period, factors including drugs used concurrently and other factors well-known in the medical field. The pharmaceutical composition of the present disclosure may be administered as an individual therapeutic agent or in combination with other therapeutic agents, and may be administered sequentially or simultaneously with a commercially available therapeutic agent. In addition, the pharmaceutical composition of the present disclosure can be single or multiple administrations. It is important to administer the minimum amount capable of obtaining the maximum effect without side effects in consideration of all the above factors, and may be easily determined by those skilled in the art. The dosage of the pharmaceutical composition of the present disclosure may be determined by an expert according to various factors such as the patient's condition, age, sex, complications, and the like. Since the active ingredient of the pharmaceutical composition of the present disclosure has excellent safety, it may be used more than the determined dose.

The term “prevention” used in the present disclosure refers to any action that inhibits or delays the occurrence, spread, or recurrence of the disease through the administration of the compound and the term “treatment” refers to any action that improves or beneficially modifies the symptoms of the disease through the administration of the compound.

In addition, in one embodiment of the present disclosure, the pharmaceutical composition may further comprise a pharmaceutically acceptable carrier, diluent, or excipient. In a particular embodiment, the invention provides a pharmaceutical composition comprising a compound represented by Chemical Formula 1, 2, 3, or 4, or a pharmaceutically acceptable salt or stereoisomer thereof, along with a pharmaceutically acceptable additive.

Examples of additives used in the above pharmaceutical compositions include sweeteners, binders, solvents, solubilizers, wetting agents, emulsifiers, dispersing agents, adsorbents, disintegrants, antioxidants, preservatives, lubricants, fillers, flavoring agents, and the like. For instance, the additives may include lactose, dextrose, sucrose, mannitol, sorbitol, cellulose, glycine, silica, talc, stearic acid, stearin, magnesium stearate, magnesium aluminosilicate, starch, gelatin, tragacanth gum, alginic acid, sodium alginate, methylcellulose, sodium carboxymethylcellulose, agar, water, ethanol, polyethylene glycol, polyvinylpyrrolidone, sodium chloride, calcium chloride, orange essence, strawberry essence, vanilla flavor, and the like.

The pharmaceutical composition can be formulated in various dosage forms for oral administration (e.g., tablets, pills, powders, capsules, syrups, or emulsions) or for parenteral administration (e.g., intramuscular, intravenous, or subcutaneous injections).

For example, the pharmaceutical composition can be formulated as an oral dosage form, wherein the additives used may include cellulose, calcium silicate, corn starch, lactose, sucrose, dextrose, calcium phosphate, stearic acid, magnesium stearate, calcium stearate, gelatin, talc, surfactants, suspending agents, emulsifiers, and diluents. Specifically, solid dosage forms for oral administration may include tablets, pills, powders, granules, and capsules. These solid forms can be formulated by mixing the composition with one or more excipients, such as starch, calcium carbonate, sucrose, lactose, and gelatin. In addition to simple excipients, lubricants such as magnesium stearate and talc may also be used. Liquid dosage forms for oral administration may include suspensions, emulsions, and syrups, which can contain various excipients in addition to commonly used diluents like water and liquid paraffin. These additional excipients may include wetting agents, sweeteners, flavorings, and preservatives.

Additionally, formulations for parenteral administration may include sterile aqueous solutions, non-aqueous solutions, suspensions, emulsions, lyophilized preparations, and suppositories. Non-aqueous solutions and suspending agents such as propylene glycol, polyethylene glycol, vegetable oils like olive oil, and injectable esters such as ethyl oleate may be utilized. Suppository bases may include Witepsol, Macrogol, Tween 61, cocoa butter, lauric fats, and glycerogelatin. Meanwhile, injectable formulations may contain conventional additives such as solvents, isotonic agents, suspending agents, emulsifiers, stabilizers, and preservatives.

Furthermore, it can be manufactured as a combination preparation with other active agents to achieve a synergistic effect of the active ingredients.

The matters mentioned in the use, composition, and treatment method of the present disclosure are equally applicable unless they are contradicting each other.

### Advantageous Effects

The heteroaryl derivative compounds of the present disclosure, a stereoisomer thereof, or a pharmaceutically acceptable salt thereof exhibit excellent inhibitory activity against kinases, particularly PKMYT1 and/or CCNE1, and as a result, can be effectively used for the treatment or prevention of diseases associated with the overactivation, amplification, or overexpression of PKMYT1 and/or CCNE1, and are particularly useful as therapeutic agents for cancer.

### BEST MODE

Hereinafter, the present disclosure is described in detail through Examples and Experimental Examples. However, the following Examples and Experimental Examples are merely presented to illustrate the present disclosure, and the scope of the present disclosure is not limited thereto.

### <Analysis and Purification Conditions>

The compounds synthesized in Examples of the present disclosure were purified by the following method or subjected to structural analysis.

### 1. LC-MS, MPLC

### LC-MS (Liquid Chromatography-Mass Spectrometry)

Agilent's 1200/G6110A equipment was used. The software used was Agilent ChemStation Rev. B. 04.03[52], and the column used a Kinetex® 5 m EVO C18 30×2.1 mm, with the column temperature set at 50° C.

Water containing 0.0375% trifluoroacetic acid was used as mobile phase A, and acetonitrile containing 0.01875% trifluoroacetic acid was used as mobile phase B.

Gradient condition (0.01 min 5% B→0.8 min 95% B→1.2 min 95% B→1.21 min 5% B→1.5 min 5% B; flow rate=1.5 mL/min); Detector: DAD (220&254 nm)

### MPLC (Medium Pressure Liquid Chromatography)

Medium pressure liquid chromatography was performed using TELEDYNE ISCO's CombiFlash Rf+UV.

### 2. NMR Analysis

The NMR analysis was recorded and analyzed using Bruker's AVANCE III 400 or AVANCE III 400 HD, and the acquired data was expressed in ppm (parts per million, δ).

The used commercially available reagents were used without further purification. In the present disclosure, the room temperature or ambient temperature refers to a temperature range of 5° C. to 40° C., for example, 10° C. to 30° C., or in another example, 20° C. to 27° C., although it is not strictly limited to the above range. For concentration under reduced pressure or solvent distillation removal, a rotary evaporator was used.

### <Preparation Example 1> Preparation of 3-(1H-Indol-2-yl)-3-oxopropanenitrile

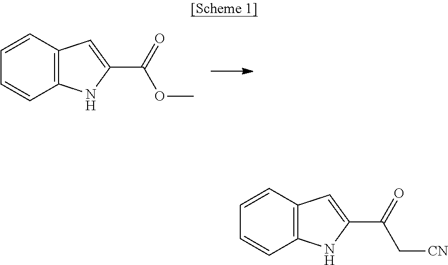

Methyl 1H-indole-2-carboxylate (5 g, 28.54 mmol) was dissolved in tetrahydrofuran (50 mL), followed by the addition of acetonitrile (6.44 g, 156.98 mmol, 8.26 mL). The reaction mixture was cooled to −78° C., and lithium bis(trimethylsilyl)amide (LiHMDS; 1 M, 85.62 mL) was slowly added dropwise under a nitrogen atmosphere and the mixture was stirred for 2 hours. The reaction mixture was then added to a saturated ammonium chloride solution (300 mL) at 0° C. and extracted with ethyl acetate (200 mL×2). The collected organic layers were washed with a saturated sodium chloride solution (200 mL), dried over with anhydrous sodium sulfate, and concentrated. The concentrated mixture was purified by MPLC (petroleum ether/ethyl acetate=1/0 to 2/1) to obtain the desired compound as a yellow solid (3 g, 16.29 mmol, 57.07% yield, MS (ESI): m/z=185.0 [M+H]⁺).

### <Preparation Example 2> Preparation of 3-(1H-benzo[d]imidazol-2-yl)-3-oxopropanenitrile

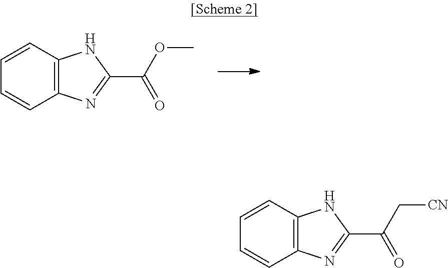

Methyl 1H-benzo[d]imidazole-2-carboxylate (3.7 g, 21.00 mmol) was dissolved in tetrahydrofuran (40 mL), followed by the addition of acetonitrile (5.17 g, 126.01 mmol, 6.63 mL). The reaction mixture was cooled to −78° C., and lithium bis(trimethylsilyl)amide (LiHMDS; 1 M, 84.01 mL) was slowly added dropwise under a nitrogen atmosphere and the mixture was stirred for 1 hour. The reaction mixture was then added to a saturated ammonium chloride solution (200 mL) at 0° C. and extracted with ethyl acetate (300 mL×2). The presence of the desired compound in the aqueous layer was confirmed by LCMS, and the mixture was purified by reverse-phase chromatography (column: Phenomenex Luna C18 150×25 mm, 10 μm; mobile phase: [water (FA)-ACN]; gradient: 5%-8% B over 10 min) to obtain the desired compound as a brown solid (1.37 g, crude, MS (ESI): m/z 186.1=[M+H]⁺; ¹H NMR (400 MHz, CDCl₃): δ=10.32 (s, 1H), 7.92 (d, J=8.4 Hz, 1H), 7.64-7.56 (m, 1H), 7.52 (t, J=7.6 Hz, 1H), 7.46-7.40 (m, 1H), 4.48 (s, 2H)).

### <Preparation Example 3> Preparation of 3-oxo-3-(4H-thieno[3,2-b]pyrrol-5-yl)propanenitrile

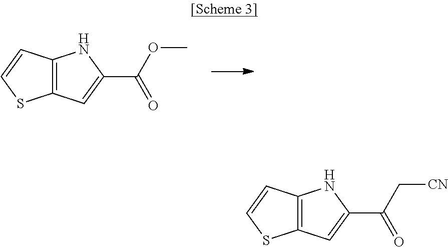

Methyl 4H-thieno[3,2-b]pyrrole-5-carboxylate (1.5 g, 8.28 mmol) was dissolved in tetrahydrofuran (20 mL), and the reaction mixture was cooled to −78° C. under a nitrogen atmosphere, lithium bis(trimethylsilyl)amide (LiHMDS; 1 M, 41.39 mL) was added slowly dropwise, and the mixture was stirred for 30 minutes. Acetonitrile (1.95 g, 47.50 mmol, 2.50 mL) was then added at −78° C. and the mixture was stirred for 3 hours at 0° C. The reaction was quenched at 0° C. with a saturated ammonium chloride solution (100 mL) and extracted with ethyl acetate (100 mL×3). The collected organic layers were washed with a saturated sodium chloride solution (200 mL), dried over with anhydrous sodium sulfate, and concentrated. The concentrated mixture was added to mixed solvent (petroleum ether/methyl tert-butyl ether/dichloromethane=2:2:1) and stirred at 25° C. for 30 minutes to obtain the desired compound as a brown solid (1.1 g, 68.46% yield).

### <Preparation Example 4> Preparation of 3-oxo-3-(4-((2-(trimethylsilyl)ethoxy)methyl)-4H-furo[3,2-b]pyrrol-5-yl)propanenitrile

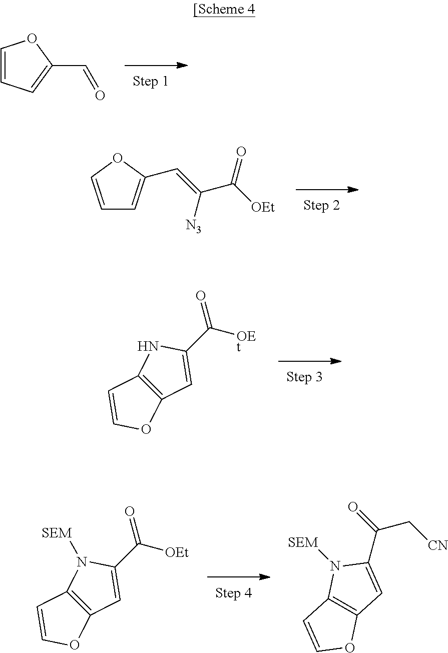 [SMILES: O=Cc1ccco1 ; CCOC(=O)/C(=C/c1ccco1)N=[N+]=[N-] ; CCOC(=O)c1cc2occc2[nH]1 ; CCOC(=O)c1cc2occc2n1COCC[Si](C)(C)C ; C[Si](C)(C)CCOCn1c(C(=O)CC#N)cc2occc21]

### [Step 1] Preparation of Ethyl (Z)-2-azido-3-(furan-2-yl)acrylate

A 20% sodium ethoxide (53.12 g, 156.11 mmol) was dissolved in ethanol (250 mL). At 0° C., furan-2-carbaldehyde (10 g, 104.08 mmol, 8.63 mL) and ethyl 2-azidoacetate (20.16 g, 156.11 mmol, 17.89 mL) were added, and the reaction mixture was stirred for 4 hours. The reaction was quenched at 0° C. with a saturated ammonium chloride solution (500 mL) and extracted with ethyl acetate (200 mL×3). The collected organic layers were washed with a saturated sodium chloride solution (200 mL), dried over with anhydrous sodium sulfate, and concentrated. The concentrated mixture was purified by MPLC (petroleum ether/ethyl acetate=1/0 to 20/1) to obtain the desired compound as a yellow oil (8 g, 37.10% yield; ¹H NMR (400 MHz, CDCl₃): δ=7.50 (d, J=2.0 Hz, 1H), 7.11 (d, J=3.6 Hz, 1H), 6.87 (s, 1H), 6.53 (dd, J=2.0, 3.6 Hz, 1H), 4.35 (q, J=7.2 Hz, 2H), 1.39 (t, J=7.2 Hz, 3H)).

### [Step 2] Preparation of Ethyl 4H-furo[3,2-b]pyrrole-5-carboxylate

The ethyl (Z)-2-azido-3-(furan-2-yl)acrylate (8 g, 38.61 mmol) prepared in Step 1 was dissolved in toluene (500 mL) and stirred at 120° C. for 12 hours. The reaction mixture was concentrated to obtain the desired compound as a gray solid, which was used directly in the subsequent reaction without further purification (6 g, 86.73% yield; ¹H NMR (400 MHz, CDCl₃): δ=8.89 (s, 1H), 7.52 (d, J=2.4 Hz, 1H), 6.81 (dd, J=0.8, 1.6 Hz, 1H), 6.46 (dd, J=0.8, 2.0 Hz, 1H), 4.36 (q, J=7.2 Hz, 2H), 1.38 (t, J=7.2 Hz, 3H)).

### [Step 3] Preparation of Ethyl 4-((2-(trimethylsilyl)ethoxy)methyl)-4H-furo[3,2-b]pyrrole-5-carboxylate

The ethyl 4H-furo[3,2-b]pyrrole-5-carboxylate (1 g, 5.58 mmol) prepared in Step 2 was dissolved in tetrahydrofuran (10 mL), then Sodium hydride (334.84 mg, 8.37 mmol, 60% purity) was slowly added at 0° C. and the mixture was stirred for 30 minutes. The 2-(trimethylsilyl)ethoxymethyl chloride (SEMCl; 1.40 g, 8.37 mmol, 1.48 mL) was added to the reaction mixture and stirred at 25° C. for 12 hours. The reaction mixture was added to cold water (50 mL) and extracted with ethyl acetate (30 mL×3). The collected organic layers were washed with saturated sodium chloride solution (20 mL), then dried over with anhydrous sodium sulfate and concentrated. The concentrated mixture was purified by MPLC (petroleum ether/ethyl acetate=1/0 to 10/1) to obtain the desired compound as a colorless oil (1.7 g, 98.44% yield; ¹H NMR (400 MHz, CDCl₃): δ=7.53 (d, J=2.4 Hz, 1H), 6.89 (s, 1H), 6.55 (d, J=2.0 Hz, 1H), 5.82 (s, 2H), 4.32 (q, J=7.2 Hz, 2H), 3.57-3.48 (m, 2H), 1.37 (t, J=7.2 Hz, 3H), 0.93-0.86 (m, 2H), 0.05 (s, 9H)).

### [Step 4] Preparation of 3-oxo-3-(4-((2-(trimethylsilyl)ethoxy)methyl)-4H-furo[3,2-b]pyrrol-5-yl)propanenitrile

The ethyl 4-((2-(trimethylsilyl)ethoxy)methyl)-4H-furo[3,2-b]pyrrole-5-carboxylate (500 mg, 1.62 mmol) prepared in Step 3 was dissolved in tetrahydrofuran (10 mL), and acetonitrile (331.66 mg, 8.08 mmol, 425.20 L) was added. Potassium bis(trimethylsilyl)amide (KHMDS; 1 M, 8.08 mL) was then slowly added at 0° C. over 30 minutes, and the mixture was stirred for 2 hours. The reaction mixture was neutralized with a saturated ammonium chloride solution (40 mL) at 0° C. and extracted with ethyl acetate (20 mL×3). The collected organic layers were washed with a saturated sodium chloride solution (50 mL), dried over with anhydrous sodium sulfate, and concentrated. The concentrated mixture was purified by MPLC (petroleum ether/ethyl acetate=1/0 to 1/1) to obtain the desired compound as a yellow solid (300 mg, 60.99% yield; ¹H NMR (400 MHz, CDCl₃): δ=7.64 (d, J=2.0 Hz, 1H), 6.85 (s, 1H), 6.59 (d, J=2.0 Hz, 1H), 5.83 (s, 2H), 3.91 (s, 2H), 3.68-3.52 (m, 2H), 1.57 (s, 1H), 1.09-0.82 (m, 2H), 0.03 (s, 9H)).

### <Preparation Example 5> Preparation of 3-(methoxymethoxy)-2,6-dimethylaniline

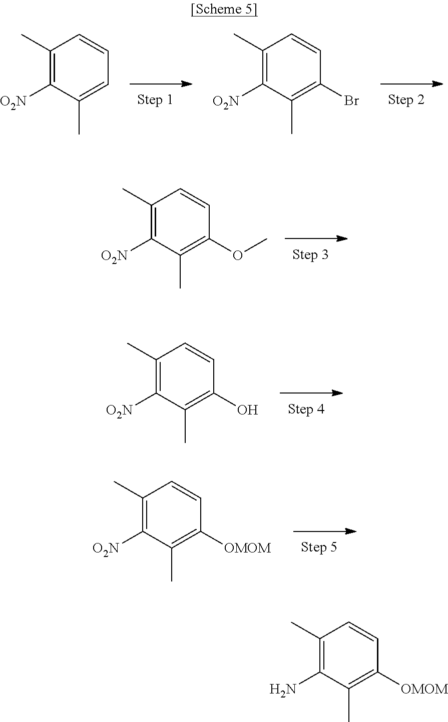

### [Step 1] Preparation of 1-bromo-2,4-dimethyl-3-nitrobenzene

1,3-dimethyl-2-nitrobenzene (20 g, 132 mmol) was dissolved in dichloromethane (66 mL), and iron (1.85 g, 33.1 mmol) and anhydrous iron(III) bromide (0.78 g, 2.65 mmol) were added. Bromine (23.26 g, 146 mmol, 7.5 mL) was then slowly added to the reaction mixture, which was stirred for 3 hours. The reaction mixture was slowly diluted with ice-cold water and the organic material was extracted with ethyl ether. The collected organic layer was washed with a 20% sodium thiosulfate solution and a saturated sodium chloride solution, dried over with sodium sulfate, and concentrated. The obtained compound was used in the subsequent reaction without further purification (30.31 g, 132 mmol, 100% yield).

### [Step 2] Preparation of 1-methoxy-2,4-dimethyl-3-nitrobenzene

The 1-bromo-2,4-dimethyl-3-nitrobenzene (30 g, 130 mmol) prepared in Step 1 was dissolved in dimethylformamide (87 mL), and copper(I) bromide (1.87 g, 13.04 mmol) was added. A solution of 25% sodium methoxide in methanol (89 mL, 391 mmol) was then slowly added to the reaction mixture, which was stirred at 95° C. for 2 hours. The reaction mixture was quenched with a saturated ammonium chloride solution and the organic material was extracted with ethyl ether. The collected organic layer was washed with a saturated sodium chloride solution, dried over with sodium sulfate, and concentrated. The concentrated mixture was purified using MPLC (hexane/ethyl acetate) to obtain the desired compound as a yellow solid (20.5 g, 113 mmol, 87% yield).

### [Step 3] Preparation of 2,4-dimethyl-3-nitrophenol

The 1-methoxy-2,4-dimethyl-3-nitrobenzene (21.81 g, 120 mmol) prepared in Step 2 was dissolved in dichloromethane (120 mL), and 1.0 M boron tribromide in dichloromethane (181 mL, 181 mmol) was slowly added at −78° C. The reaction mixture was stirred at room temperature for 16 hours, then slowly added with a saturated sodium bicarbonate solution at 0° C. The organic material was extracted with dichloromethane. The collected organic layer was washed with a saturated sodium chloride solution, dried over with sodium sulfate, and concentrated. The obtained compound was used in the subsequent reaction without further purification. (19.52 g, 117 mmol, 97% yield).

### [Step 4] Preparation of 1-(methoxymethoxy)-2,4-dimethyl-3-nitrobenzene

The 2,4-dimethyl-3-nitrophenol (20.38 g, 122 mmol) prepared in Step 3 was dissolved in dichloromethane (406 mL), and N,N-diisopropylethylamine (39.4 g, 305 mmol, 53.2 mL) was added. Chloromethyl methyl ether (9.81 g, 122 mmol, 9.26 mL) was slowly added to the reaction mixture, which was then stirred at room temperature for 2 hours. Dichloromethane was added to the reaction mixture, which was then washed with water and a 0.1 M hydrochloric acid solution. The organic layer was dried over with sodium sulfate and concentrated. The concentrated mixture was purified using MPLC (hexane/ethyl acetate) to obtain the desired compound as a yellow solid (24.41 g, 116 mmol, 95% yield).

### [Step 5] Preparation of 3-(methoxymethoxy)-2,6-dimethylaniline

The 1-(methoxymethoxy)-2,4-dimethyl-3-nitrobenzene (24.41 g, 116 mmol) prepared in Step 4 was dissolved in methanol (385 mL) and Pd/C (2.46 g, 2.31 mmol) was added, and the resulting mixture was stirred under a hydrogen atmosphere for 16 hours. After confirming the desired compound using LCMS, the reaction mixture was filtered through celite and concentrated. The resulting compound was used in the next reaction without further purification (18.93 g, 104 mmol, 90% yield, MS (ESI): m/z 182 [M+H]⁺).

### <Example 1 and 2> Preparation of (S)-(6-amino-5-(3-hydroxy-2,6-dimethylphenyl)-5H-pyrrolo[2,3-b]pyrazin-7-yl)(1H-indol-2-yl)methanone and (R)-(6-amino-5-(3-hydroxy-2,6-dimethylphenyl)-5H-pyrrolo[2,3-b]pyrazin-7-yl)(1H-indol-2-yl)methanone

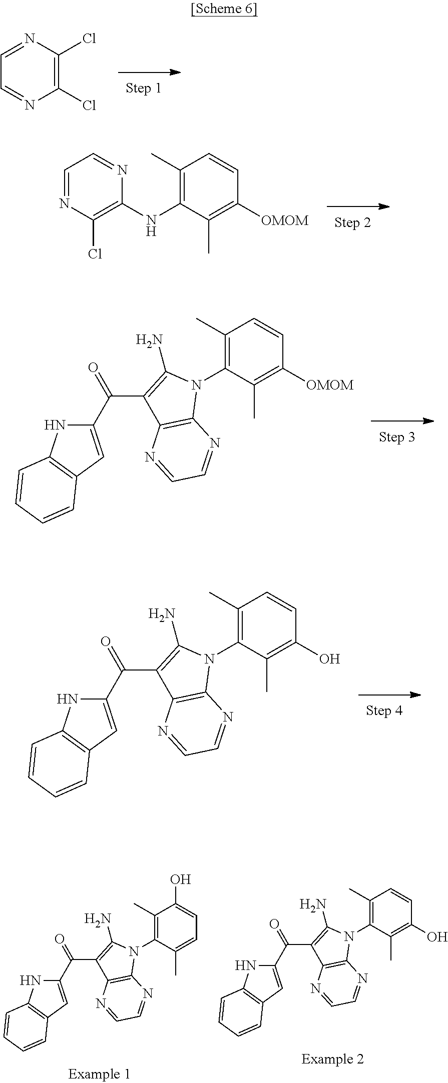

### [Step 1] Preparation of 3-chloro-N-(3-(methoxymethoxy)-2,6-dimethylphenyl)pyrazin-2-amine

2,3-Dichloropyrazine (1.64 g, 11.04 mmol) and 3-(methoxymethoxy)-2,6-dimethylaniline (2 g, 11.04 mmol) were dissolved in toluene (20 mL), and Potassium tert-butoxide (t-BuOK; 1.86 g, 16.55 mmol), Xantphos (319.27 mg, 551.78 mol), and Pd₂(dba)₃ (1.01 g, 1.10 mmol) were then added, and the reaction mixture was stirred at 80° C. for 12 hours. After confirming the completion of the reaction by LCMS, the reaction mixture was concentrated. The concentrated mixture was purified by MPLC (petroleum ether/ethyl acetate=1/0 to 5/1) and further purified by prep-HPLC (column: Phenomenex luna C18 150*40 mm*15 μm; mobile phase: [water (FA)-ACN]; gradient: 40%-70% B over 15 minutes) to yield the desired compound as a yellow oil (500 mg, 1.62 mmol, 14.65%, MS (ESI): m/z=294.0 [M+H]⁺).

### [Step 2] Preparation of (6-amino-5-(3-(methoxymethoxy)-2,6-dimethylphenyl)-5H-pyrrolo[2,3-b]pyrazin-7-yl)(1H-indol-2-yl)methanone

3-(1H-Indole-2-yl)-3-oxopropanenitrile (62.70 mg, 340.43 mol) prepared in Preparation Example 1 was dissolved in dioxane (5 mL) and Sodium tert-butoxide (t-BuONa; 98.15 mg, 1.02 mmol) was added at 0° C., and the reaction mixture was stirred for 30 minutes. 3-chloro-N-(3-(methoxymethoxy)-2,6-dimethylphenyl)pyrazin-2-amine (100 mg, 340.43 mol) prepared in Step 1 and BINAP Pd G3 (33.82 mg, 34.04 mol) were then added, and the reaction mixture was stirred at 100° C. for 12 hours. The reaction mixture was concentrated, and the residue was purified by prep-TLC (SiO₂, petroleum ether/ethyl acetate=1/1) to obtain the desired compound as a yellow oil (30 mg, 57.76 mol, 16.97% yield; ¹H NMR (400 MHz, DMSO-d₆): δ=13.80 (s, 1H), 9.14-8.49 (m, 1H), 8.44 (d, J=3.2 Hz, 1H), 7.96 (d, J=2.8 Hz, 1H), 7.88 (s, 1H), 7.72 (s, 1H), 7.68 (d, J=8.4 Hz, 1H), 7.36-7.20 (m, 3H), 7.12 (t, J=7.6 Hz, 1H), 3.44 (s, 3H), 1.88 (s, 3H), 1.84 (s, 3H)).

### [Step 3] Preparation of (6-amino-5-(3-hydroxy-2,6-dimethylphenyl)-5H-pyrrolo[2,3-b]pyrazin-7-yl)(1H-indol-2-yl)methanone

(6-amino-5-(3-(methoxymethoxy)-2,6-dimethylphenyl)-5H-pyrrolo[2,3-b]pyrazin-7-yl)(1H-indol-2-yl)methanone (30 mg, 57.76 mol) prepared in Step 2 was dissolved in methanol (2 mL) and hydrochloric acid (0.6 mL) was added, and the reaction mixture was stirred at 25° C. for 2 hours. The reaction mixture was then concentrated, and the residue was purified by prep-HPLC (column: Phenomenex Luna C18 250×50 mm, 15 μm; mobile phase: [water (formic acid)-acetonitrile]; gradient: 52% A-82% B over 9 minutes) to obtain the desired compound as a yellow solid (10 mg, 25.16 mol, 43.56% yield; ¹H NMR (400 MHz, DMSO-d₆): δ=13.84 (s, 1H), 9.68 (s, 1H), 8.42 (d, J=2.8 Hz, 1H), 7.96 (d, J=3.2 Hz, 1H), 7.84 (d, J=1.2 Hz, 1H), 7.72 (d, J=8.0 Hz, 1H), 7.68 (d, J=8.4 Hz, 1H), 7.31-7.26 (m, 1H), 7.15-7.07 (m, 2H), 6.96 (d, J=8.4 Hz, 1H), 1.84 (s, 3H), 1.76 (s, 3H)).

### [Step 4] Preparation of (S)-(6-amino-5-(3-hydroxy-2,6-dimethylphenyl)-5H-pyrrolo[2,3-b]pyrazin-7-yl)(1H-indol-2-yl)methanone and (R)-(6-amino-5-(3-hydroxy-2,6-dimethylphenyl)-5H-pyrrolo[2,3-b]pyrazin-7-yl)(1H-indol-2-yl)methanone

(6-amino-5-(3-hydroxy-2,6-dimethylphenyl)-5H-pyrrolo[2,3-b]pyrazin-7-yl)(1H-indol-2-yl)methanone (95 mg, 239.04 mol) obtained in Step 3 was separated and purified using supercritical fluid chromatography (SFC; column: 250 mm×30 mm, 10 μm; mobile phase A: CO₂, 50%; mobile phase B: EtOH (0.1% NH₃·H₂O), 50%; isocratic elution mode) to obtain two desired compounds.
Example 1 ((S)-(6-amino-5-(3-hydroxy-2,6-dimethylphenyl)-5H-pyrrolo[2,3-b]pyrazin-7-yl)(1H-indol-2-yl)methanone): Peak 2, RT=0.996 min, (43.1 mg, 42.70% yield, 94.12% purity, yellow solid). MS (ESI): m/z=398.1 [M+H]⁺; ¹H NMR (400 MHz, METHANOL-d₄): δ=9.20 (d, J=2.8 Hz, 1H), 8.76 (d, J=2.8 Hz, 1H), 8.52 (d, J=8.4 Hz, 1H), 8.44 (d, J=8.4 Hz, 1H), 8.36 (s, 1H), 8.12 (t, J=7.6 Hz, 1H), 7.97-7.89 (m, 2H), 7.76 (d, J=8.4 Hz, 1H), 2.72 (s, 3H), 2.68 (s, 3H).
Example 2 ((R)-(6-amino-5-(3-hydroxy-2,6-dimethylphenyl)-5H-pyrrolo[2,3-b]pyrazin-7-yl)(1H-indol-2-yl)methanone): Peak 1, RT=0.614 min, (41.2 mg, 41.60% yield, 95.92% purity, yellow solid). MS (ESI): m/z=398.1 [M+H]⁺; ¹H NMR (400 MHz, METHANOL-d₄): δ=8.42 (d, J=1.6 Hz, 1H), 7.96 (d, J=2.8 Hz, 1H), 7.72 (d, J=8.2 Hz, 1H), 7.64 (d, J=8.2 Hz, 1H), 7.56 (s, 1H), 7.32 (t, J=7.5 Hz, 1H), 7.17-7.06 (m, 2H), 6.96 (br d, J=8.1 Hz, 1H), 1.92 (s, 3H), 1.87 (s, 3H).

SFC analysis method: column: Chiralpak IC-3 50×4.6 mm I.D., 3 um; mobile phase: mobile phase A is CO₂, mobile phase B is EtOH (0.05% DEA); Gradient elution: 40% EtOH (0.05% DEA) in CO₂; flow rate: 3 mL/min; Detector: PDA; column temperature: 35° C.; Back Pressure: 100 Bar

### <Example 11 and 12> Preparation of (S)-(2-amino-1-(3-hydroxy-2,6-dimethylphenyl)-5,6-dimethyl-1H-pyrrolo[2,3-b]pyridin-3-yl)(1H-benzo[d]imidazol-2-yl)methanone and (R)-(2-amino-1-(3-hydroxy-2,6-dimethylphenyl)-5,6-dimethyl-1H-pyrrolo[2,3-b]pyridin-3-yl)(1H-benzo[d]imidazol-2-yl)methanone

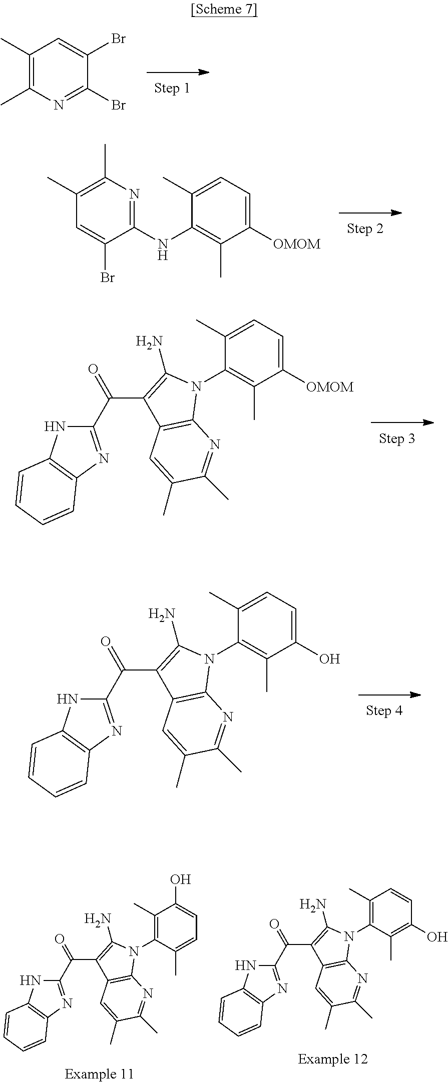

### [Step 1] Preparation of 3-bromo-N-(3-(methoxymethoxy)-2,6-dimethylphenyl)-5,6-dimethylpyridine-2-amine

3-(methoxymethoxy)-2,6-dimethylaniline (1 g, 5.52 mmol) prepared in Preparation Example 5 was dissolved in toluene (50 mL) and to this solution were added 2,3-dibromo-5,6-dimethylpyridine (1.61 g, 6.07 mmol), Xantphos (319.27 mg, 551.78 mol), cesium carbonate (Cs₂CO₃; 5.39 g, 16.55 mmol), and Pd₂(dba)₃ (505.28 mg, 551.78 mol) and the reaction mixture was stirred at 100° C. for 12 hours. After concentrating the reaction mixture, it was purified by MPLC (petroleum ether/ethyl acetate=10/1˜3/1) to obtain the desired compound as a yellow solid (1.5 g, 3.82 mmol, 69.21% yield, MS (ESI): m/z=365.1 [M+H]⁺).

### [Step 2] Preparation of (2-amino-1-(3-(methoxymethoxy)-2,6-dimethylphenyl)-5,6-dimethyl-1H-pyrrolo[2,3-b]pyridin-3-yl)(1H-benzo[d]imidazol-2-yl)methanone

3-(1H-benzo[d]imidazole-2-yl)-3-oxopropanenitrile (38.02 mg, 205.33 mol) prepared in Preparation Example 2 and 3-bromo-N-(3-(methoxymethoxy)-2,6-dimethylphenyl)-5,6-dimethylpyridin-2-amine (50 mg, 136.89 mol) prepared in Step 1 were dissolved in dioxane (1 mL). To this solution were added BINAP Pd G3 (13.60 mg, 13.69 mol) and potassium phosphate (K₃PO₄; 87.17 mg, 410.66 mol) and the reaction mixture was stirred at 100° C. for 12 hours. After concentrating the reaction mixture, it was purified by MPLC (petroleum ether/ethyl acetate=10/1˜1/1) to obtain the desired compound as a yellow solid (50 mg, 15.56% yield, MS (ESI): m/z=470.2 [M+H]⁺).

### [Step 3] Preparation of (2-amino-1-(3-hydroxy-2,6-dimethylphenyl)-5,6-dimethyl-1H-pyrrolo[2,3-b]pyridin-3-yl)(1H-benzo[d]imidazol-2-yl)methanone

(2-amino-1-(3-(methoxymethoxy)-2,6-dimethylphenyl)-5,6-dimethyl-1H-pyrrolo[2,3-b]pyridin-3-yl)(1H-benzo[d]imidazol-2-yl)methanone (74 mg, 157.60 mol) prepared in Step 2 was dissolved in methanol (3 mL). To this solution, HCl/MeOH (4 M, 3 mL) was added, and the mixture was stirred at 25° C. for 1 hour. After concentrating the reaction mixture, it was purified using a reverse-phase column (under formic acid conditions) to obtain the desired compound as a yellow solid (20 mg, 47.01 mol, 29.83% yield, MS (ESI): m/z=426.1 [M+H]⁺).

### [Step 4] Preparation of (S)-(2-amino-1-(3-hydroxy-2,6-dimethylphenyl)-5,6-dimethyl-1H-pyrrolo[2,3-b]pyridin-3-yl)(1H-benzo[d]imidazol-2-yl)methanone and (R)-(2-amino-1-(3-hydroxy-2,6-dimethylphenyl)-5,6-dimethyl-1H-pyrrolo[2,3-b]pyridin-3-yl)(1H-benzo[d]imidazol-2-yl)methanone

(2-amino-1-(3-hydroxy-2,6-dimethylphenyl)-5,6-dimethyl-1H-pyrrolo[2,3-b]pyridin-3-yl)(1H-benzo[d]imidazol-2-yl)methanone (20 mg, 47.01 mol) obtained in Step 3 was separated and purified using supercritical fluid chromatography (SFC; column: 250 mm×30 mm, 10 μm; mobile phase A: CO₂, 70%, MeOH (0.1% NH₃H₂O) 30%; isocratic elution mode) to obtain two desired compounds.
Example 11 ((S)-(2-amino-1-(3-hydroxy-2,6-dimethylphenyl)-5,6-dimethyl-1H-pyrrolo[2,3-b]pyridin-3-yl)(1H-benzo[d]imidazol-2-yl)methanone): Peak 2, RT=2.112 min, (8.4 mg, 42.00% yield, 100% purity, white solid). MS (ESI): m/z=426.1 [M+H]⁺; ¹H NMR (400 MHz, METHANOL-d₄): δ=8.72-8.41 (m, 1H), 7.76 (dd, J=2.8, 6.0 Hz, 2H), 7.48 (s, 2H), 7.36-7.21 (m, 2H), 7.04 (d, J=8.4 Hz, 1H), 2.48 (s, 3H), 2.36 (s, 3H), 1.92 (s, 3H), 1.88 (s, 3H).
Example 12 ((R)-(2-amino-1-(3-hydroxy-2,6-dimethylphenyl)-5,6-dimethyl-1H-pyrrolo[2,3-b]pyridin-3-yl)(1H-benzo[d]imidazol-2-yl)methanone): Peak 1, RT=2.004 min, (6.8 mg, 34.0% yield, 100% purity, white solid). MS (ESI): m/z=426.1 [M+H]⁺; ¹H NMR (400 MHz, METHANOL-d₄): δ=8.66-8.35 (m, 1H), 7.72 (dd, J=2.8, 6.0 Hz, 2H), 7.44 (d, J=8.4 Hz, 2H), 7.30-7.15 (m, 2H), 7.00 (d, J=8.4 Hz, 1H), 2.44 (s, 3H), 2.32 (s, 3H), 1.88 (s, 3H), 1.84 (s, 3H).

SFC analysis method: column: Chiralcel OD-3 50*4.6 mm I.D., 3 um; mobile phase: mobile phase A is CO₂, mobile phase B is MeOH (0.05% DEA); Gradient elution: MeOH (0.05% DEA) in CO₂ from 5% to 40%, Flow rate: 3 mL/min; Detector: PDA; column temperature: 35° C.; Back Pressure: 100 Bar

### <Example 1> to <Example 50>

All other Example compounds (Examples 3 to 10, and Examples 13 to 50) of the present disclosure were prepared in a similar manner to Examples 1, 2, 11, and 12, and the compound names, chemical structures, NMR, and LCMS analysis results for each Example compound are summarized in Table 1 below.

TABLE 1

| | | | | LC- |
|---|---|---|---|---|
| | | | | MS |
| | | | | [M + |
| Example | Structure | Compound Name | ¹H NMR | H]⁺ |
| 1 | 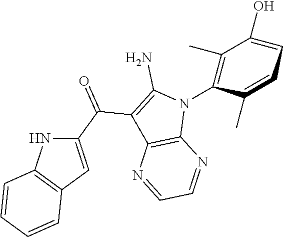 | (S)-(6-amino-5-(3- hydroxy-2,6- dimethylphenyl)- 5H-pyrrolo[2,3- b]pyrazin-7-yl)(1H- indol-2- yl)methanone | ¹H NMR (400 MHz, MeOD) δ = 9.20 (d, J = 2.8 Hz, 1H), 8.76 (d, J = 2.8 Hz, 1H), 8.52 (d, J = 8.4 Hz, 1H), 8.44 (d, J = 8.4 Hz, 1H), 8.36 (s, 1H), 8.12 (t, J = 7.6 Hz, 1H), 7.97- 7.89 (m, 2H), 7.76 (d, J = 8.4 Hz, 1H), 2.72 (s, 3H), 2.68 (s, 3H). | 398.1 |
| 2 | 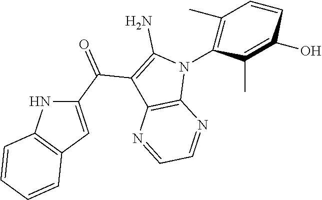 | (R)-(6-amino-5-(3- hydroxy-2,6- dimethylphenyl)- 5H-pyrrolo[2,3- b]pyrazin-7-yl)(1H- indol-2- yl)methanone | ¹H NMR (400 MHz, MeOD) δ = 8.42 (d, J = 1.6 Hz, 1H), 7.96 (d, J = 2.8 Hz, 1H), 7.72 (d, J = 8.2 Hz, 1H), 7.64 (d, J = 8.2 Hz, 1H), 7.56 (s, 1H), 7.32 (t, J = 7.5 Hz, 1H), 7.17- 7.06 (m, 2H), 6.96 (br d, J = 8.1 Hz, 1H), 1.92 (s, 3H), 1.87 (s, 3H). | 398.1 |
| 3 | 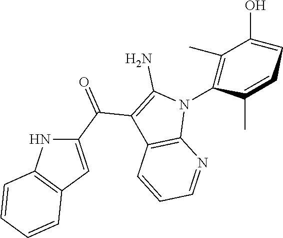 | (S)-(2-amino-1-(3- hydroxy-2,6- dimethylphenyl)- 1H-pyrrolo[2,3- b]pyridin-3-yl)(1H- benzo[d]imidazol- 2-yl)methanone | ¹H NMR (400 MHz, MeOD) δ = 8.85-8.64 (m, 1H), 7.89 (d, J = 4.8 Hz, 1H), 7.82 (d, J = 7.6 Hz, 1H), 7.67 (d, J = 8.0 Hz, 1H), 7.46-7.31 (m, 1H), 7.19 (d, J = 8.0 Hz, 1H), 7.00 (d, J = 8.4 Hz, 1H), 1.96 (s, 1H), 1.91 (s, 1H). | 398.1 |
| 4 | 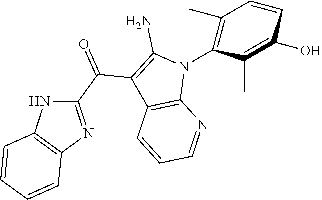 | (R)-(2-amino-1-(3- hydroxy-2,6- dimethylphenyl)- 1H-pyrrolo[2,3- b]pyridin-3-yl)(1H- benzo[d]imidazol- 2-yl)methanone | ¹H NMR (400 MHz, MeOD) δ = 8.73 (d, J = 2.4 Hz, 1H), 7.87 (d, J = 4.0 Hz, 1H), 7.72 (s, 2H), 7.36 (s, 2H), 7.16 (d, J = 7.6 Hz, 2H), 6.97 (d, J = 8.0 Hz, 1H), 1.91 (d, J = 16.4 Hz, 6H). | 398.1 |
| 5 | 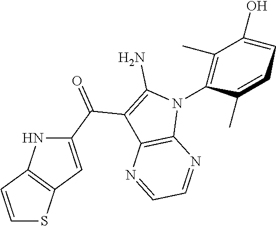 | (S)-(6-amino-5-(3- hydroxy-2,6- dimethylphenyl)- 5H-pyrrolo[2,3- b]pyrazin-7-yl)(4H- thieno[3,2-b]pyrrol- 5-yl)methanone | ¹H NMR (400 MHz, MeOD) δ = 8.35 (d, J = 3.2 Hz, 1H), 7.93 (d, J = 3.2 Hz, 1H), 7.45 (s, 1H), 7.44 (d, J = 5.2 Hz, 1H), 7.19 (d, J = 5.2 Hz, 1H), 7.12 (d, J = 8.4 Hz, 1H), 6.94 (d, J = 8.4 Hz, 1H), 1.90 (s, 3H), 1.86 (s, 3H). | 404.1 |
| 6 | 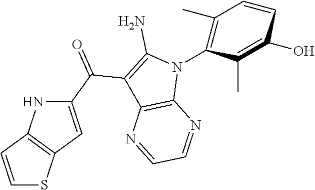 | (R)-(6-amino-5-(3- hydroxy-2,6- dimethylphenyl)- 5H-pyrrolo[2,3- b]pyrazin-7-yl)(4H- thieno[3,2-b]pyrrol- 5-yl)methanone | ¹H NMR (400 MHz, MeOD) δ = 8.35 (d, J = 3.2 Hz, 1H), 7.92 (d, J = 3.2 Hz, 1H), 7.44 (s, 1H), 7.43 (d, J = 5.2 Hz, 1H), 7.19 (d, J = 5.2 Hz, 1H), 7.09 (d, J = 8.4 Hz, 1H), 6.92 (d, J = 8.4 Hz, 1H), 1.88 (s, 3H), 1.85 (s, 3H). | 404.1 |
| 7 | 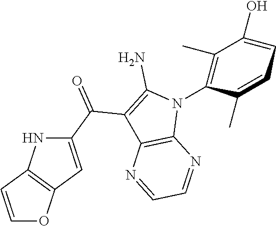 | (S)-(6-amino-5-(3- hydroxy-2,6- dimethylphenyl)- 5H-pyrrolo[2,3- b]pyrazin-7-yl)(4H- furo[3,2-b]pyrrol-5- yl)methanone | ¹H NMR (400 MHz, MeOD) δ = 8.33 (d, J = 3.2 Hz, 1H), 7.92 (d, J = 3.2 Hz, 1H), 7.67 (d, J = 2.4 Hz, 1H), 7.15 (d, J = 8.4 Hz, 1H), 7.06 (s, 1H), 6.97 (d, J = 8.4 Hz, 1H), 6.70 (d, J = 2.4 Hz, 1H), 1.91 (s, 3H), 1.86 (s, 3H). | 388.1 |
| 8 | 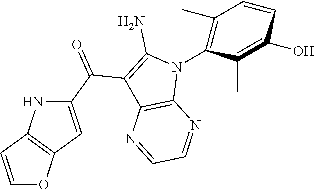 | (R)-(6-amino-5-(3- hydroxy-2,6- dimethylphenyl)- 5H-pyrrolo[2,3- b]pyrazin-7-yl)(4H- furo[3,2-b]pyrrol-5- yl)methanone | ¹H NMR (400 MHz, MeOD) δ = 8.33 (d, J = 3.2 Hz, 1H), 7.92 (d, J = 3.2 Hz, 1H), 7.67 (d, J = 2.4 Hz, 1H), 7.15 (d, J = 8.4 Hz, 1H), 7.06 (s, 1H), 6.97 (d, J = 8.4 Hz, 1H), 6.70 (d, J = 2.4 Hz, 1H), 1.91 (s, 3H), 1.86 (s, 3H). | 388.1 |
| 9 | 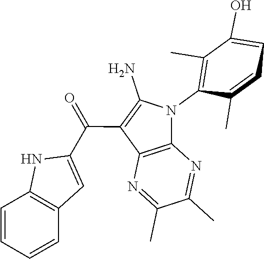 | (S)-(6-amino-5-(3- hydroxy-2,6- dimethylphenyl)- 2,3-dimethyl-5H- pyrrolo[2,3- b]pyrazin-7-yl)(1H- indol-2- yl)methanone | ¹H NMR (400 MHz, CDCl₃) δ = 15.00 (s, 1H), 7.80 (d, J = 8.0 Hz, 1H), 7.65 (d, J = 8.4 Hz, 1H), 7.55 (s, 1H), 7.36 (t, J = 7.6 Hz, 1H), 7.17 (t, J = 7.6 Hz, 1H), 6.97 (d, J = 8.4 Hz, 1H), 6.71 (d, J = 8.4 Hz, 1H), 2.83 (s, 3H), 2.60 (s, 3H), 1.95 (s, 3H), 1.93 (s, 3H). | 426.1 |
| 10 | 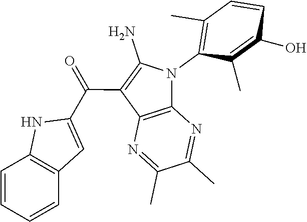 | (R)-(6-amino-5-(3- hydroxy-2,6- dimethylphenyl)- 2,3-dimethyl-5H- pyrrolo[2,3- b]pyrazin-7-yl)(1H- indol-2- yl)methanone | ¹H NMR (400 MHz, CDCl₃) δ = 14.99 (s, 1H), 7.80 (d, J = 8.0 Hz, 1H), 7.65 (d, J = 8.0 Hz, 1H), 7.56 (s, 1H), 7.36 (t, J = 7.6 Hz, 1H), 7.17 (t, J = 7.6 Hz, 1H), 6.95 (d, J = 8.4 Hz, 1H), 6.68 (d, J = 8.4 Hz, 1H), 2.83 (s, 3H), 2.61 (s, 3H), 1.96 (s, 3H), 1.93 (s, 3H). | 426.1 |
| 11 | 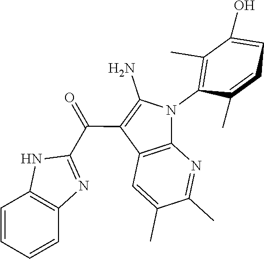 | (S)-(2-amino-1-(3- hydroxy-2,6- dimethylphenyl)- 5,6-dimethyl-1H- pyrrolo[2,3- b]pyridin-3-yl)(1H- benzo[d]imidazol- 2-yl)methanone | ¹H NMR (400 MHz, MeOD) δ = 8.72-8.41 (m, 1H), 7.76 (dd, J = 2.8, 6.0 Hz, 2H), 7.48 (s, 2H), 7.36- 7.21 (m, 2H), 7.04 (d, J = 8.4 Hz, 1H), 2.48 (s, 3H), 2.36 (s, 3H), 1.92 (s, 3H), 1.88 (s, 3H). | 426.1 |
| 12 | 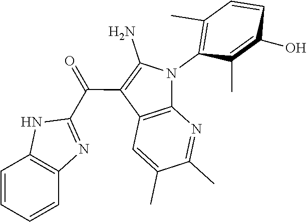 | (R)-(2-amino-1-(3- hydroxy-2,6- dimethylphenyl)- 5,6-dimethyl-1H- pyrrolo[2,3- b]pyridin-3-yl)(1H- benzo[d]imidazol- 2-yl)methanone | ¹H NMR (400 MHz, MeOD) δ = 8.66-8.35 (m, 1H), 7.72 (dd, J = 2.8, 6.0 Hz, 2H), 7.44 (d, J = 8.4 Hz, 2H), 7.30-7.15 (m, 2H), 7.00 (d, J = 8.4 Hz, 1H), 2.44 (s, 3H), 2.32 (s, 3H), 1.88 (s, 3H), 1.84 (s, 3H). | 426.1 |
| 13 | 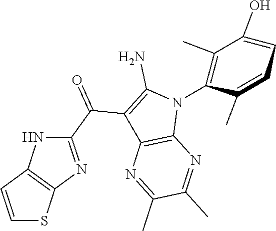 | (S)-(6-amino-5-(3- hydroxy-2,6- dimethylphenyl)- 2,3-dimethyl-5H- pyrrolo[2,3- b]pyrazin-7-yl)(4H- thieno[3,2-b]pyrrol- 5-yl)methanone | ¹H NMR (400 MHz, CDCl₃) δ = 7.49 (s, 1H), 7.37 (d, J = 5.2 Hz, 1H), 7.14 (d, J = 5.2 Hz, 1H), 6.94 (d, J = 7.6 Hz, 1H), 6.68 (d, J = 8.4 Hz, 1H), 3.50 (s, 2H), 2.77 (s, 3H), 2.59 (s, 3H), 1.94 (s, 3H), 1.91 (s, 3H). | 432.1 |
| 14 | 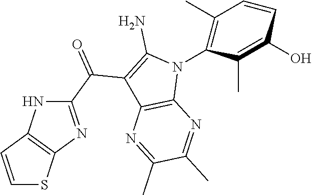 | (R)-(6-amino-5-(3- hydroxy-2,6- dimethylphenyl)- 2,3-dimethyl-5H- pyrrolo[2,3- b]pyrazin-7-yl)(4H- thieno[3,2-b]pyrrol- 5-yl)methanone | ¹H NMR (400 MHz, CDCl₃) δ = 7.48 (s, 1H), 7.37 (d, J = 5.2 Hz, 1H), 7.14 (d, J = 5.2 Hz, 1H), 6.97 (d, J = 7.6 Hz, 1H), 6.71 (d, J = 8.4 Hz, 1H), 3.50 (s, 2H), 2.76 (s, 3H), 2.57 (s, 3H), 1.93 (s, 3H), 1.92 (s, 3H). | 432.1 |
| 15 | 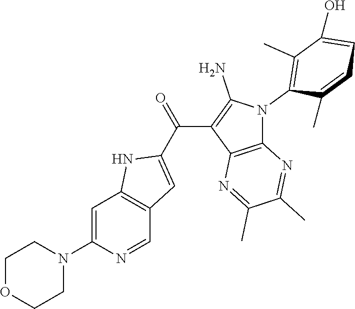 | (S)-(6-amino-5-(3- hydroxy-2,6- dimethylphenyl)- 2,3-dimethyl-5H- pyrrolo[2,3- b]pyrazin-7-yl)(6- morpholino-1H- pyrrolo[3,2- c]pyridin-2- yl)methanone | ¹H NMR (400 MHz, DMSO-d₆) δ = 8.68 (s, 1H), 8.31-8.21 (m, 1H), 7.95- 7.80 (m, 2H), 7.08 (d, J = 8.0 Hz, 1H), 6.94 (d, J = 8.0 Hz, 1H), 6.85 (s, 1H), 3.80- 3.72 (m, 4H), 3.45-3.42 (m, 4H), 1.82 (s, 3H), 1.75 (s, 3H). | 512.2 |
| 16 | 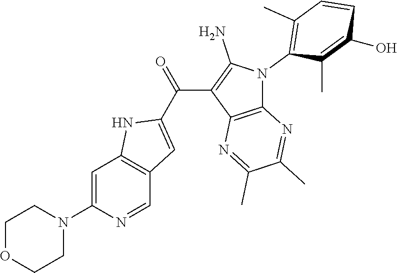 | (R)-(6-amino-5-(3- hydroxy-2,6- dimethylphenyl)- 2,3-dimethyl-5H- pyrrolo[2,3- b]pyrazin-7-yl)(6- morpholino-1H- pyrrolo[3,2- c]pyridin-2- yl)methanone | ¹H NMR (400 MHz, DMSO-d₆) δ = 8.67 (s, 1H), 8.28-8.18 (m, 1H), 7.87- 7.81 (m, 2H), 7.08 (d, J = 8.0 Hz, 1H), 6.93 (d, J = 7.6 Hz, 1H), 6.85 (s, 1H), 3.80- 3.74 (m, 4H), 3.46-3.42 (m, 4H), 1.82 (s, 3H), 1.75 (s, 3H). | 512.2 |
| 17 | 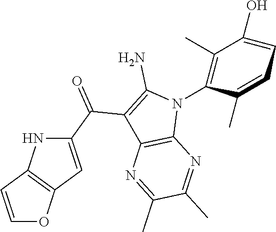 | (S)-(6-amino-5-(3- hydroxy-2,6- dimethylphenyl)- 2,3-dimethyl-5H- pyrrolo[2,3- b]pyrazin-7-yl)(4H- furo[3,2-b]pyrrol-5- yl)methanone | ¹H NMR (400 MHz, MeOD) δ = 7.66 (d, J = 2.4 Hz, 1H), 7.08 (d, J = 8.4 Hz, 1H), 7.00 (d, J = 0.8 Hz, 1H), 6.91 (d, J = 8.4 Hz, 1H), 6.70 (dd, J = 0.8, 2.0 Hz, 1H), 2.71 (s, 3H), 2.47 (s, 3H), 1.88 (s, 3H), 1.84 (s, 3H). | 416.2 |
| 18 | 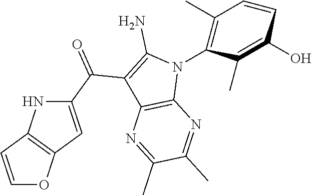 | (R)-(6-amino-5-(3- hydroxy-2,6- dimethylphenyl)- 2,3-dimethyl-5H- pyrrolo[2,3- b]pyrazin-7-yl)(4H- furo[3,2-b]pyrrol-5- yl)methanone | ¹H NMR (400 MHz, MeOD) δ = 7.66 (d, J = 2.4 Hz, 1H), 7.07 (d, J = 8.4 Hz, 1H), 7.00 (d, J = 0.8 Hz, 1H), 6.90 (d, J = 8.4 Hz, 1H), 6.70 (dd, J = 0.8, 2.0 Hz, 1H), 2.71 (s, 3H), 2.47 (s, 3H), 1.87 (s, 3H), 1.84 (s, 3H). | 416.2 |
| 19 | 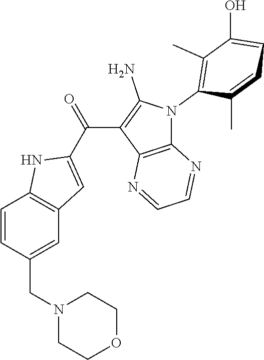 | (S)-(6-amino-5-(3- hydroxy-2,6- dimethylphenyl)- 5H-pyrrolo[2,3- b]pyrazin-7-yl)(5- (morpholinomethyl)- 1H-indol-2- yl)methanone | ¹H NMR (400 MHz, DMSO-d₆) δ = 13.82 (s, 1H), 9.66 (s, 1H), 9.11-8.45 (m, 2H), 8.41 (d, J = 2.8 Hz, 1H), 7.98 (d, J = 2.8 Hz, 1H), 7.80 (s, 1H), 7.66- 7.58 (m, 2H), 7.33-7.21 (m, 1H), 7.13 (d, J = 8.4 Hz, 1H), 6.98 (d, J = 8.0 Hz, 1H), 3.62-3.50 (m, 6H), 2.45-2.31 (m, 4H), 1.83 (s, 3H), 1.75 (s, 3H). | 497.2 |
| 20 | 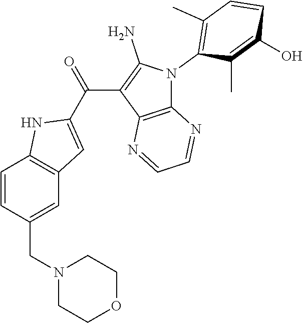 | (R)-(6-amino-5-(3- hydroxy-2,6- dimethylphenyl)- 5H-pyrrolo[2,3- b]pyrazin-7-yl)(5- (morpholinomethyl)- 1H-indol-2- yl)methanone | ¹H NMR (400 MHz, DMSO-d₆) δ = 13.82 (s, 1H), 9.66 (s, 1H), 9.18- 8.46 (m, 2H), 8.41 (d, J = 2.8 Hz, 1H), 7.98 (d, J = 3.2 Hz, 1H), 7.81 (s, 1H), 7.70- 7.53 (m, 2H), 7.30-7.24 (m, 1H), 7.13 (d, J = 8.0 Hz, 1H), 6.98 (d, J = 8.0 Hz, 1H), 3.68-3.48 (m, 6H), 2.46-2.34 (m, 4H), 1.83 (s, 3H), 1.75 (s, 3H). | 497.2 |
| 21 | 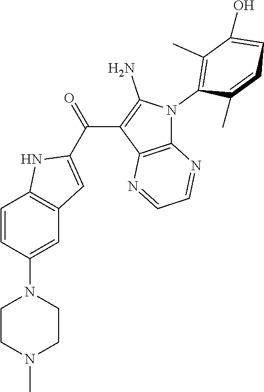 | (S)-(6-amino-5-(3- hydroxy-2,6- dimethylphenyl)- 5H-pyrrolo[2,3- b]pyrazin-7-yl)(5- (4-methylpiperazin- 1-yl)-1H-indol-2- yl)methanone | ¹H NMR (400 MHz, DMSO-d₆) δ = 13.81 (s, 1H), 9.69 (s, 1H), 8.40 (d, J = 2.8 Hz, 1H), 7.97 (d, J = 2.8 Hz, 1H), 7.64 (s, 1H), 7.56 (d, J = 8.8 Hz, 1H), 7.17-7.07 (m, 3H), 6.98 (d, J = 8.4 Hz, 1H), 3.16-3.05 (m, 4H), 2.64-2.53 (m, 4H), 2.24 (s, 3H), 1.82 (s, 3H), 1.75 (s, 3H). | 496.2 |
| 22 | 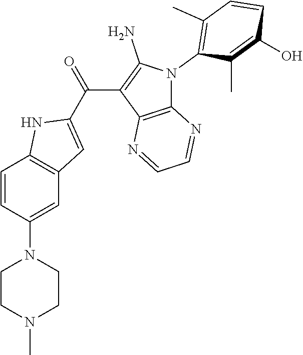 | (R)-(6-amino-5-(3- hydroxy-2,6- dimethylphenyl)- 5H-pyrrolo[2,3- b]pyrazin-7-yl)(5- (4-methylpiperazin- 1-yl)-1H-indol-2- yl)methanone | ¹H NMR (400 MHz, DMSO-d₆) δ = 13.81 (s, 1H), 9.71 (s, 1H), 8.40 (d, J = 2.8 Hz, 1H), 7.97 (d, J = 2.8 Hz, 1H), 7.64 (s, 1H), 7.56 (d, J = 8.8 Hz, 1H), 7.16-7.07 (m, 3H), 6.98 (d, J = 8.0 Hz, 1H), 3.16-3.06 (m, 4H), 2.63-2.52 (m, 4H), 2.24 (s, 3H), 1.82 (s, 3H), 1.75 (s, 3H). | 496.2 |
| 23 | 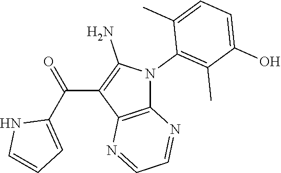 [SMILES: Cc1ccc(O)c(C)c1-n1c(N)c(C(=O)c2ccc[nH]2)c2nccnc21] | (6-amino-5-(3- hydroxy-2,6- dimethylphenyl)- 5H-pyrrolo[2,3- b]pyrazin-7-yl)(1H- pyrrol-2- yl)methanone | ¹H NMR (400 MHz, DMSO-d₆) δ 14.46 (s, 1H), 8.21 (s, 1H), 7.86 (s, 1H), 7.24 (s, 1H), 7.20 (s, 1H), 7.07 (dd, J = 8.6, 3.9 Hz, 1H), 6.94 (dd, J = 8.4, 3.1 Hz, 1H), 6.29 (dd, J = 3.9, 2.1 Hz, 1H), 1.80 (s, 3H), 1.72 (s, 3H). | 348.1 |
| 24 | 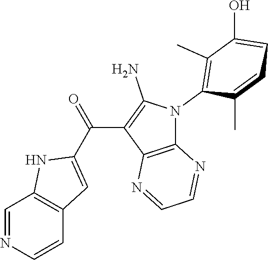 | (S)-(6-amino-5-(3- hydroxy-2,6- dimethylphenyl)- 5H-pyrrolo[2,3- b]pyrazin-7-yl)(1H- pyrrolo[2,3- c]pyridin-2- yl)methanone | ¹H NMR (400 MHz, DMSO-d₆) δ = 14.46 (s, 1H), 9.71 (s, 1H), 9.17 (s, 1H), 8.44 (d, J = 3.2 Hz, 1H), 8.22 (d, J = 4.4 Hz, 1H), 8.03 (d, J = 3.2 Hz, 1H), 7.88 (d, J = 1.2 Hz, 1H), 7.78 (d, J = 5.2 Hz, 1H), 7.14 (d, J = 8.4 Hz, 1H), 7.00 (d, J = 8.4 Hz, 1H), 1.84 (s, 3H), 1.76 (s, 3H). | 399.1 |
| 25 | 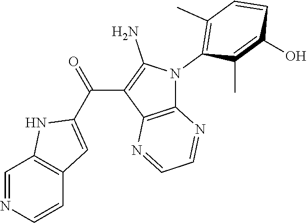 | (R)-(6-amino-5-(3- hydroxy-2,6- dimethylphenyl)- 5H-pyrrolo[2,3- b]pyrazin-7-yl)(1H- pyrrolo[2,3- c]pyridin-2- yl)methanone | ¹H NMR (400 MHz, DMSO-d₆) δ = 14.33 (s, 1H), 9.70 (s, 1H), 9.12 (s, 1H), 8.44 (d, J = 3.2 Hz, 1H), 8.19 (d, J = 4.4 Hz, 1H), 8.02 (d, J = 3.2 Hz, 1H), 7.86 (d, J = 1.2 Hz, 1H), 7.72 (d, J = 5.2 Hz, 1H), 7.13 (d, J = 8.4 Hz, 1H), 6.99 (d, J = 8.4 Hz, 1H), 1.83 (s, 3H), 1.76 (s, 3H). | 399.1 |
| 26 | 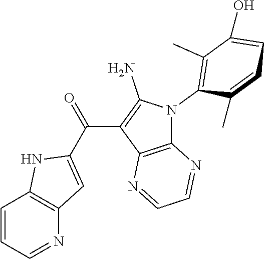 | (S)-(6-amino-5-(3- hydroxy-2,6- dimethylphenyl)- 5H-pyrrolo[2,3- b]pyrazin-7-yl)(1H- pyrrolo[3,2- b]pyridin-2- yl)methanone | ¹H NMR (400 MHz, MeOD) δ = 8.45 (d, J = 4.0 Hz, 1H), 8.41 (d, J = 4.0 Hz, 1H), 8.18 (d, J = 8.0 Hz, 1H), 7.99 (d, J = 4.0 Hz, 1H), 7.71 (s, 1H), 7.36 (m, 1H), 7.17 (d, J = 8.0 Hz, 1H), 6.99 (d, J = 8.0 Hz, 1H), 1.93 (s, 3H), 1.88 (s, 3H). | 399.1 |
| 27 | 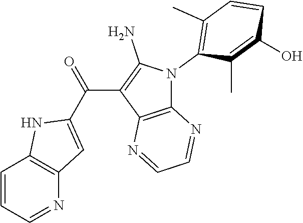 | (R)-(6-amino-5-(3- hydroxy-2,6- dimethylphenyl)- 5H-pyrrolo[2,3- b]pyrazin-7-yl)(1H- pyrrolo[3,2- b]pyridin-2- yl)methanone | ¹H NMR (400 MHz, MeOD) δ = 8.45 (d, J = 4.0 Hz, 1H), 8.41 (d, J = 4.0 Hz, 1H), 8.18 (d, J = 8.0 Hz, 1H), 7.98 (d, J = 4.0 Hz, 1H), 7.71 (s, 1H), 7.36 (m, 1H), 7.17 (d, J = 8.0 Hz, 1H), 6.98 (d, J = 8.0 Hz, 1H), 1.92 (s, 3H), 1.88 (s, 3H). | 399.1 |
| 28 | 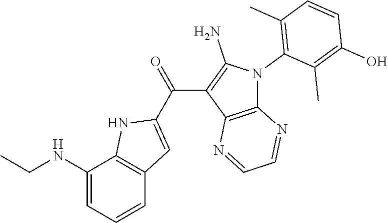 [SMILES: CCNc1cccc2cc(C(=O)c3c(N)n(-c4c(C)ccc(O)c4C)c4nccnc34)[nH]c12] | (6-amino-5-(3- hydroxy-2,6- dimethylphenyl)- 5H-pyrrolo[2,3- b]pyrazin-7-yl)(7- (ethylamino)-1H- indol-2- yl)methanone | | 441.2 |
| 29 | 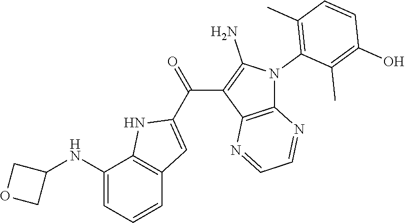 [SMILES: Cc1ccc(O)c(C)c1-n1c(N)c(C(=O)c2cc3cccc(NC4COC4)c3[nH]2)c2nccnc21] | (6-amino-5-(3- hydroxy-2,6- dimethylphenyl)- 5H-pyrrolo[2,3- b]pyrazin-7-yl)(7- (oxetan-3- ylamino)-1H-indol- 2-yl)methanone | | 469.2 |
| 30 | 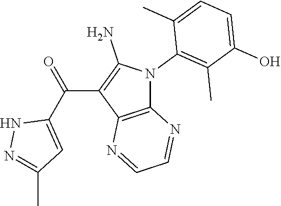 [SMILES: Cc1cc(C(=O)c2c(N)n(-c3c(C)ccc(O)c3C)c3nccnc23)[nH]n1] | (6-amino-5-(3- hydroxy-2,6- dimethylphenyl)- 5H-pyrrolo[2,3- b]pyrazin-7-yl)(3- methyl-1H-pyrazol- 5-yl)methanone | | 363.1 |
| 31 | 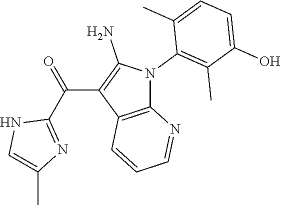 [SMILES: Cc1c[nH]c(C(=O)c2c(N)n(-c3c(C)ccc(O)c3C)c3ncccc23)n1] | (2-amino-1-(3- hydroxy-2,6- dimethylphenyl)- 1H-pyrrolo[2,3- b]pyridin-3-yl)(4- methyl-1H- imidazol-2- yl)methanone | | 362.2 |
| 32 | 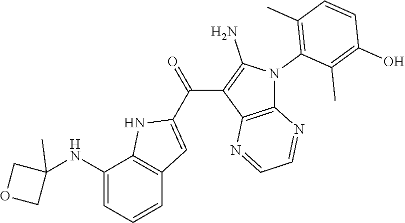 [SMILES: Cc1ccc(O)c(C)c1-n1c(N)c(C(=O)c2cc3cccc(NC4(C)COC4)c3[nH]2)c2nccnc21] | (6-amino-5-(3- hydroxy-2,6- dimethylphenyl)- 5H-pyrrolo[2,3- b]pyrazin-7-yl)(7- ((3-methyloxetan- 3-yl)amino)-1H- indol-2- yl)methanone | | 483.2 |
| 33 | 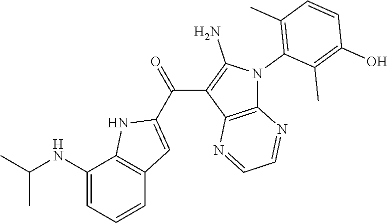 [SMILES: Cc1ccc(O)c(C)c1-n1c(N)c(C(=O)c2cc3cccc(NC(C)C)c3[nH]2)c2nccnc21] | (6-amino-5-(3- hydroxy-2,6- dimethylphenyl)- 5H-pyrrolo[2,3- b]pyrazin-7-yl)(7- (isopropylamino)- 1H-indol-2- yl)methanone | | 455.2 |
| 34 | 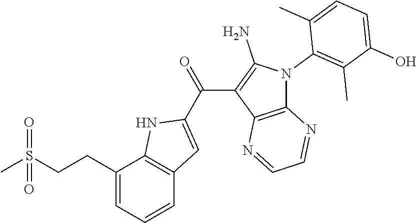 [SMILES: Cc1ccc(O)c(C)c1-n1c(N)c(C(=O)c2cc3cccc(CCS(C)(=O)=O)c3[nH]2)c2nccnc21] | (6-amino-5-(3- hydroxy-2,6- dimethylphenyl)- 5H-pyrrolo[2,3- b]pyrazin-7-yl)(7- (2- (methylsulfonyl) ethyl)-1H-indol-2- yl)methanone | | 504.2 |
| 35 | 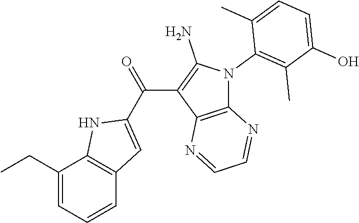 [SMILES: CCc1cccc2cc(C(=O)c3c(N)n(-c4c(C)ccc(O)c4C)c4nccnc34)[nH]c12] | (6-amino-5-(3- hydroxy-2,6- dimethylphenyl)- 5H-pyrrolo[2,3- b]pyrazin-7-yl)(7- ethyl-1H-indol-2- yl)methanone | | 426.2 |
| 36 | 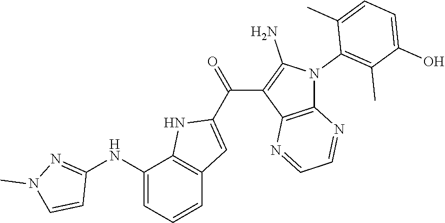 [SMILES: Cc1ccc(O)c(C)c1-n1c(N)c(C(=O)c2cc3cccc(Nc4ccn(C)n4)c3[nH]2)c2nccnc21] | (6-amino-5-(3- hydroxy-2,6- dimethylphenyl)- 5H-pyrrolo[2,3- b]pyrazin-7-yl)(7- ((1-methyl-1H- pyrazol-3- yl)amino)-1H- indol-2- yl)methanone | | 493.2 |
| 37 | 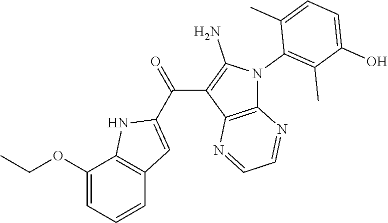 [SMILES: CCOc1cccc2cc(C(=O)c3c(N)n(-c4c(C)ccc(O)c4C)c4nccnc34)[nH]c12] | (6-amino-5-(3- hydroxy-2,6- dimethylphenyl)- 5H-pyrrolo[2,3- b]pyrazin-7-yl)(7- ethoxy-1H-indol-2- yl)methanone | | 442.2 |
| 38 | 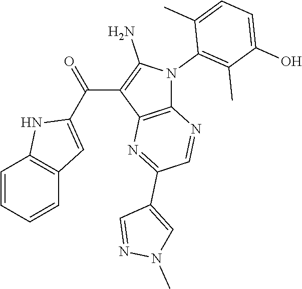 [SMILES: Cc1ccc(O)c(C)c1-n1c(N)c(C(=O)c2cc3ccccc3[nH]2)c2nc(-c3cnn(C)c3)cnc21] | (6-amino-5-(3- hydroxy-2,6- dimethylphenyl)-2- (1-methyl-1H- pyrazol-4-yl)-5H- pyrrolo[2,3- b]pyrazin-7-yl)(1H- indol-2- yl)methanone | | 478.2 |
| 39 | 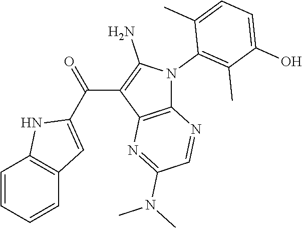 [SMILES: Cc1ccc(O)c(C)c1-n1c(N)c(C(=O)c2cc3ccccc3[nH]2)c2nc(N(C)C)cnc21] | (6-amino-2- (dimethylamino)-5- (3-hydroxy-2,6- dimethylphenyl)- 5H-pyrrolo[2,3- b]pyrazin-7-yl)(1H- indol-2- yl)methanone | | 441.2 |
| 40 | 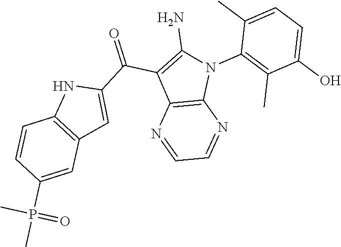 [SMILES: Cc1ccc(O)c(C)c1-n1c(N)c(C(=O)c2cc3cc(P(C)(C)=O)ccc3[nH]2)c2nccnc21] | (6-amino-5-(3- hydroxy-2,6- dimethylphenyl)- 5H-pyrrolo[2,3- b]pyrazin-7-yl)(5- (dimethyl- phosphoryl)- 1H-indol-2- yl)methanone | ¹H NMR (400 MHz, DMSO-d₆) δ 13.96 (s, 1H), 9.70 (s, 1H), 8.43 (d, J = 3.1 Hz, 1H), 8.20 (d, J = 12.7 Hz, 1H), 8.04 (t, J = 1.4 Hz, 1H), 8.00 (d, J = 3.0 Hz, 1H), 7.81 (d, J = 8.5 Hz, 1H), 7.65 (ddd, J = 10.0, 8.4, 1.4 Hz, 1H), 7.14 (d, J = 8.3 Hz, 1H), 6.99 (d, J = 8.3 Hz, 1H), 1.83 (s, 3H), 1.76 (s, 3H), 1.71 (s, 3H), 1.68 (s, 3H). | 474.2 |
| 41 | 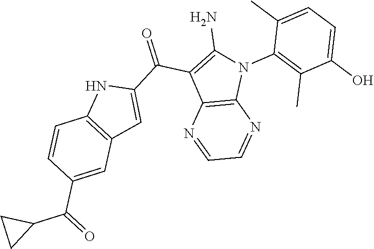 [SMILES: Cc1ccc(O)c(C)c1-n1c(N)c(C(=O)c2cc3cc(C(=O)C4CC4)ccc3[nH]2)c2nccnc21] | (6-amino-5-(3- hydroxy-2,6- dimethylphenyl)- 5H-pyrrolo[2,3- b]pyrazin-7-yl)(5- (cyclopropane- carbonyl)- 1H-indol-2- yl)methanone | | 466.2 |
| 42 | 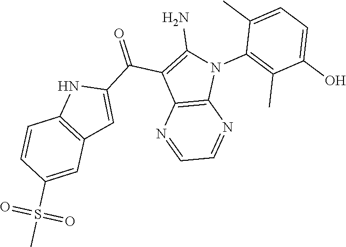 [SMILES: Cc1ccc(O)c(C)c1-n1c(N)c(C(=O)c2cc3cc(S(C)(=O)=O)ccc3[nH]2)c2nccnc21] | (6-amino-5-(3- hydroxy-2,6- dimethylphenyl)- 5H-pyrrolo[2,3- b]pyrazin-7-yl)(5- (methylsulfonyl)- 1H-indol-2- yl)methanone | | 476.1 |
| 43 | 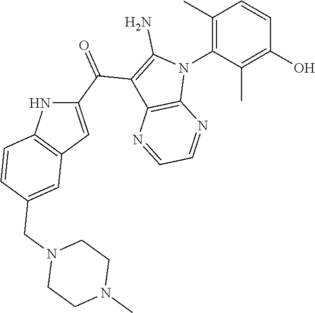 [SMILES: Cc1ccc(O)c(C)c1-n1c(N)c(C(=O)c2cc3cc(CN4CCN(C)CC4)ccc3[nH]2)c2nccnc21] | (6-amino-5-(3- hydroxy-2,6- dimethylphenyl)- 5H-pyrrolo[2,3- b]pyrazin-7-yl)(5- ((4- methylpiperazin-1- yl)methyl)-1H- indol-2- yl)methanone | | 510.3 |
| 44 | 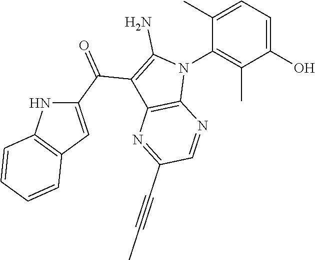 [SMILES: CC#Cc1cnc2c(n1)c(C(=O)c1cc3ccccc3[nH]1)c(N)n2-c1c(C)ccc(O)c1C] | (6-amino-5-(3- hydroxy-2,6- dimethylphenyl)-2- (prop-1-yn-1-yl)- 5H-pyrrolo[2,3- b]pyrazin-7-yl)(1H- indol-2- yl)methanone | | 436.2 |
| 45 | 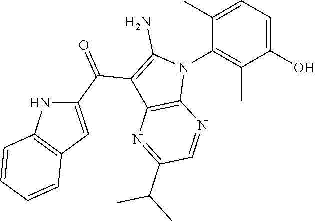 [SMILES: Cc1ccc(O)c(C)c1-n1c(N)c(C(=O)c2cc3ccccc3[nH]2)c2nc(C(C)C)cnc21] | (6-amino-5-(3- hydroxy-2,6- dimethylphenyl)-2- isopropyl-5H- pyrrolo[2,3- b]pyrazin-7-yl)(1H- indol-2- yl)methanone | | 440.2 |
| 46 | 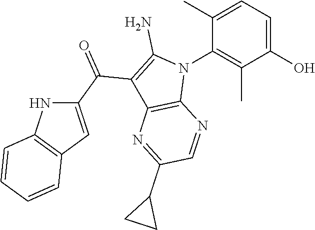 [SMILES: Cc1ccc(O)c(C)c1-n1c(N)c(C(=O)c2cc3ccccc3[nH]2)c2nc(C3CC3)cnc21] | (6-amino-2- cyclopropyl-5-(3- hydroxy-2,6- dimethylphenyl)- 5H-pyrrolo[2,3- b]pyrazin-7-yl)(1H- indol-2- yl)methanone | | 438.2 |
| 47 | 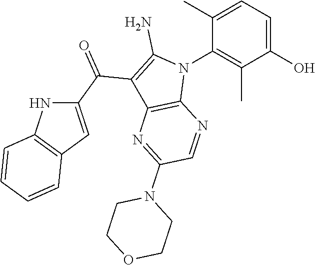 [SMILES: Cc1ccc(O)c(C)c1-n1c(N)c(C(=O)c2cc3ccccc3[nH]2)c2nc(N3CCOCC3)cnc21] | (6-amino-5-(3- hydroxy-2,6- dimethylphenyl)-2- morpholino-5H- pyrrolo[2,3- b]pyrazin-7- yl)(1H- indol-2- yl)methanone | | 483.2 |
| 48 | 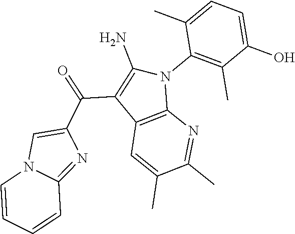 [SMILES: Cc1cc2c(C(=O)c3cn4ccccc4n3)c(N)n(-c3c(C)ccc(O)c3C)c2nc1C] | (2-amino-1-(3- hydroxy-2,6- dimethylphenyl)- 5,6-dimethyl-1H- pyrrolo[2,3- b]pyridin-3- yl)(imidazo[1,2- a]pyridin-2- yl)methanone | | 426.2 |
| 49 | 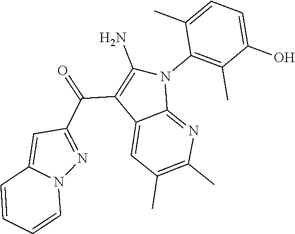 [SMILES: Cc1cc2c(C(=O)c3cc4ccccn4n3)c(N)n(-c3c(C)ccc(O)c3C)c2nc1C] | (2-amino-1-(3- hydroxy-2,6- dimethylphenyl)- 5,6-dimethyl-1H- pyrrolo[2,3- b]pyridin-3- yl)(pyrazolo[1,5- a]pyridin-2- yl)methanone | | 426.2 |
| 50 | 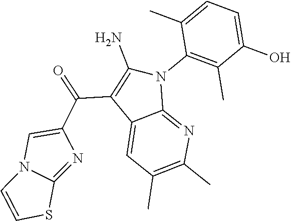 [SMILES: Cc1cc2c(C(=O)c3cn4ccsc4n3)c(N)n(-c3c(C)ccc(O)c3C)c2nc1C] | (2-amino-1-(3- hydroxy-2,6- dimethylphenyl)- 5,6-dimethyl-1H- pyrrolo[2,3- b]pyridin-3- yl)(imidazo[2,1- b]thiazol-6- yl)methanone | | 432.1 |

### Experimental Example 1: Evaluation by Thermal Shift Analysis

The degree of compound binding to Myt1-KD was tested using a Thermal Shift Assay. Each protein was diluted to a concentration of 2 μM in 50 mM Tris (pH 7.5) with 100 μM of the compound. SYPRO™ Orange dye was added to the solution, and the plate was transferred to a QuantStudio3 Real-Time PCR System (Applied Biosystems by Thermo Fisher Scientific). The temperature was gradually increased from 25° C. to 99° C. at a rate of 0.1° C. per second, and the melting temperature (Tm) was calculated using Protein Thermal Shift software (version 1.3) (Applied Biosystems, Grand Island, NY). The degree of protein melting in the presence of the compound compared to DMSO was calculated according to the following Equation 1.

ΔTm=Tm (Temperature of Example compound)−Tm (Temperature of DMSO) [Equation 1]

The ΔTm value shows a significant correlation with cellular activity, and the results are presented in Table 2 below.

TABLE 2

| Example Compound | ΔTm (° C.) |
|---|---|
| 1 | A |
| 2 | B |
| 3 | A |
| 4 | B |
| 5 | A |
| 6 | B |
| 7 | A |
| 8 | B |
| 9 | A |
| 10 | B |
| 11 | A |
| 12 | B |
| 13 | A |
| 14 | B |
| 15 | A |
| 16 | B |
| 17 | B |
| 18 | A |
| 19 | A |
| 20 | B |
| 21 | B |
| 22 | A |
| 23 | B |
| 24 | B |
| 25 | A |
| 26 | A |
| 27 | B |
| 40 | A |

A: ΔTm (° C.) ≥ 5° C.;
B: 5° C. ≥ ΔTm (° C.)

### Experimental Example 2: Evaluation of Inhibitory Activity Against PKMYT1 Kinase

To evaluate the inhibitory activity of the compounds synthesized according to the above Examples against PKMYT1 kinase, the Example compounds were reacted with purified human PKMYT1 enzyme and assessed using the following method.

The reaction buffer was used as a composition of 200 mM Tris-HCl pH 7.4, 100 mM MgCl₂, 0.5 mg/mL BSA, 0.25 mM DTT, and the reactions of all the tests were carried out on the reaction buffer.

The compound was diluted in 12 steps using a serial dilution method from a 10 mM DMSO stock, and the enzyme activity was measured at a final compound concentration of 10˜0.00005645 μM. During the test, the enzyme activity was confirmed using in vitro ADP-Glo™kinase assay (Promega) after reacting with the proper concentration of PKMYT1 enzyme, purified ATP, and enzyme substrate (CDK1) at 25° C. for 1 hour. At a ratio of 2:2:1, an enzyme-active reaction solution, an ADP-Glo reaction solution, and an enzyme-active detection solution were reacted to measure the inhibition of the enzyme's activity with luminescence. Based on the fluorescence of the enzyme activity of the solvent control that was not treated with the compound, the degree of enzyme activity inhibition was calculated according to the treatment concentration of each compound, and the concentration of each compound that inhibits 50% of enzyme activity was determined as IC₅₀ (nM) value and the value was obtained by using GraphPad Prism 8.3.0 (GraphPad software Inc., San Diego) software. The results are shown in Table 3 below.

TABLE 3

| Example Compound | IC₅₀ (nM) |
|---|---|
| 1 | A |
| 3 | A |
| 5 | A |
| 7 | A |
| 9 | B |
| 13 | A |
| 15 | A |
| 18 | A |
| 19 | A |
| 22 | A |
| 23 | A |
| 25 | A |

A: IC₅₀ ≤ 200 nM;
B: 200 nM < IC₅₀ ≤ 500 nM;
C: 500 nM < IC₅₀

The results from the above Experimental Examples demonstrate that the compounds of the present disclosure exhibit high inhibitory activity against PKMYT1 and effectively suppress the proliferation of cells with activated PKMYT1. Furthermore, it was confirmed that the compounds of the present disclosure also exhibit significant inhibitory activity against cells with CCNE1 overactivation, amplification, or overexpression.

The present disclosure has been described in detail through preferred Examples and Experimental Examples, however, the scope of the present disclosure is not limited to the specific compounds disclosed in these Examples and should be interpreted according to the appended claims. Furthermore, those skilled in the art will understand that many modifications and variations can be made without departing from the scope of the present disclosure.

1. A compound represented by the Chemical Formula 1 below, a stereoisomer thereof, or a pharmaceutically acceptable salt thereof: 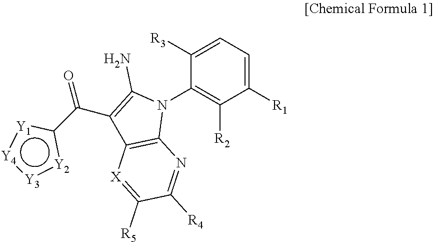
in the Chemical Formula 1 above,
R₁ to R₃ are each independently —C₁₋₆ alkyl or —OH;
R₄ and R₅ are each independently —H, —C₁₋₆ alkyl, —C₂₋₆ alkenyl, —C₂₋₆ alkynyl, —C₁₋₆ aminoalkyl, —C₁₋₆ hydroxyalkyl, —C₁₋₆ haloalkyl, —CN, —NH(C₁₋₆ alkyl), —N(C₁₋₆ alkyl)(C₁₋₆ alkyl), —O—(C₁₋₆ alkyl), 3-6 membered cycloalkyl, 3-12 membered heterocycloalkyl, phenyl, or 5-6 membered heteroaryl {wherein one or more H atoms in the ring of the 3-6 membered cycloalkyl, 3-12 membered heterocycloalkyl, phenyl, or 5-6 membered heteroaryl may be substituted with —C₁₋₆ alkyl};
X is CH or N;
Y₁ and Y₂ are each independently NH, N, or —CR_{Y1};
Y₃ and Y₄ are each independently N or CR_{Y2}, or Y₃ and Y₄ are bonded together to form an 8-9 membered aromatic fused ring {wherein the 8-9 membered aromatic fused ring may include one or more N, O, or S atoms within the ring; and one or more H atoms in the 8-9 membered aromatic fused ring may be substituted with —C₁₋₆ alkyl, —C₁₋₆ aminoalkyl, —C₁₋₆ hydroxyalkyl, —C₁₋₆ haloalkyl, —(CH₂)m-Rₐ, —NH—R_{b}, —O—R_{c}, or -halo};
R_{Y1} and R_{Y2} are each independently —H, —C₁₋₆ alkyl, or —C₁₋₆ alkenyl;
m is 0, 1, 2, 3, or 4;
Rₐ is —S(═O)₂—C₁₋₆ alkyl, —P(═O)—(C₁₋₆ alkyl)(C₁₋₆ alkyl), 3-12 membered heterocycloalkyl, or —C(═O)-(3-6 membered cycloalkyl) {wherein one or more H atoms in the 3-12 membered heterocycloalkyl or —C(═O)-(3-6 membered cycloalkyl) ring may be substituted with —C₁₋₆ alkyl, —O—C₁₋₆ alkyl, —C₁₋₆ haloalkyl, or -halo}; and
R_{b} and R_{c} are each independently —C₁₋₆ alkyl, —C₁₋₆ haloalkyl, —S(═O)₂—C₁₋₆ alkyl, 3-6 membered heterocycloalkyl, or 5-6 membered heteroaryl {wherein one or more H atoms in the 3-6 membered heterocycloalkyl or 5-6 membered heteroaryl ring may be substituted with —C₁₋₆ alkyl, —O—C₁₋₆ alkyl, —C₁₋₆ haloalkyl, or -halo}.

2. A pharmaceutical composition for preventing or treating cancer, comprising the compound according to claim 1, the stereoisomers thereof, or the pharmaceutically acceptable salt thereof as an active ingredient.

3. A method for preventing or treating cancer, the method comprising administering to a subject in need thereof the compound according to claim 1, the stereoisomers thereof, or the pharmaceutically acceptable salt thereof as an active ingredient.
